(19) United States
(12) Patent Application Publication
(10) Pub. No.: US 2017/0340777 A1
(43) Pub. Date: Nov. 30, 2017

(54) Shape Memory Alloy Orthopedic Implant

(71) Applicant: The Texas A&M University System, College Station, TX (US)

(72) Inventors: Ji Ma, Bryan, TX (US); Mukund I. Gundanna, College Station, TX (US); Ibrahim Karaman, College Station, TX (US); Arun R. Srinivasa, College Station, TX (US)

(73) Assignee: The Texas A&M University System, College Station, TX (US)

(21) Appl. No.: 15/525,972

(22) PCT Filed: Nov. 13, 2015
(86) PCT No.: PCT/US2015/060718

§ 371 Date: May 11, 2017

Related U.S. Application Data
(60) Provisional application No. 62/079,959, filed on Nov. 14, 2014.

(51) Int. Cl. A61L 27/50; A61L 27/06; A61F 2/44; A61L 27/04; A61F 2/30

(52) U.S. Cl. CPC A61L 27/50; A61F 2/4465; A61L 27/047; A61F 2/442; A61L 27/06; A61F 2002/30978; A61F 2002/3097; A61F 2002/30095; A61F 2002/4435; A61F 2310/00095; A61L 2400/16; A61L 2430/38; A61F 2310/00023

(57) ABSTRACT

Systems and methods discussed herein provide for fabricating orthopedic implants one or more shape-memory alloys including TiNi and TiNb and shape-setting the alloys to the geometry appropriate for the orthopedic implant. The shape-setting may include tuning the transformation temperature of the one or more alloys, and a single implant may comprise one or more alloys that may differ in composition, shape-setting process, or both.

### CROSS-REFERENCE TO RELATED APPLICATIONS

This application is a 35 U.S.C. §371 national stage application of PCT/US2015/060718 filed Nov. 13, 2015, and entitled “Shape Memory Alloy Orthopedic Implant,” which claims benefit of U.S. Provisional Application Ser. No. 62/079,959 filed Nov. 14, 2014, and entitled “Shape Memory Alloy Orthopedic Implant,” each of which is hereby incorporated herein by reference in its entirety.

### STATEMENT REGARDING FEDERALLY SPONSORED RESEARCH OR DEVELOPMENT

This invention was made with government support under grant number NSF-IiP Grant No. 1355529 awarded by the National Science Foundation, Division of Industrial Innovation and Partnerships. The government has certain rights in the invention.

### BACKGROUND

The present disclosure relates generally to the field of orthopedic medical implants, and more particularly relates to implants made of a shape memory alloys and methods for replacing structures such as intervertebral discs. Still more particularly, the present disclosure relates to the use of shape memory alloys to manufacture biocompatible orthopedic medical implants that combines strength with flexibility to guard against stress shielding, loosening, complications during surgery and post-surgery, and other potential failures in order to increase the life and safety of the implant.

The human spine serves as the main structural weight bearing support of the human skeleton. The spinal column comprises a plurality of vertebrae separated by intervertebral discs. Moving down the spinal column, five distinct regions exist as follows: cervical, thoracic, lumbar, sacral, and coccygeal. The cervical region comprises seven vertebra; the thoracic region, twelve; the lumbar region, five; the sacral, five; and the coccygeal, four. The cervical, thoracic, and lumbar vertebrae generally remain separate throughout an individual's lifetime whereas the sacral and coccygeal vertebrae fuse to form the sacrum and coccyx, respectively. In general, each vertebra consists of an anterior, cancellous, portion and a posterior arch comprising two pedicles and two laminae which support seven processes (four articular, two transverse, and one spinous). The spinal cord runs through a passageway between the anterior and posterior portions of the vertebrae.

When vertebrae and/or intervertebral discs become compromised by trauma, disease, or degeneration, the ability for the spinal column to effectively serve its weight bearing support function is diminished. Furthermore, the spinal cord or nerve roots may subsequently become impinged. As a result, an individual may experience debilitating pain, loss of spinal column stability, and/or reduced range of motion (i.e. flexion, rotation, and extension) of the spinal column. To alleviate these issues, removal and replacement of compromised intervertebral discs is often required if other non-invasive methods (i.e. drug treatment or physical therapy) prove unavailing.

Recent techniques to some degree have taken into consideration the natural balancing of the body. In these, spinal deformity correction surgery is aimed at reestablishing normal physiological coronal and sagittal balance, and preserving natural motion of the body. However, even these more recent procedures still fail to take in account many aspects for providing optimal techniques consistent with the body's natural balance and anatomy.

Even with efforts toward minimally invasive surgeries for the replacement of intervertebral discs, the operation is still intrusive and requires recovery time and in some cases rehabilitation. Implants may need to be replaced due wear, loosening, or biocompatibility issues. One type of wear is stress shielding which is the reduction in bone density due to the removal of normal stress from the bone by an orthopedic implant such as an intervertebral disc implant.

Surgeries to install such orthopedic implants may be intrusive and require time in recovery as well as the expense of surgery and recovery. Surgeries may also be complicated by size and shape changes made to an orthopedic implant by a surgeon during implantation such as for example, turning screws or keys associated with the implant of the installation of the implant onto other implant members, such as guiderails.

### BRIEF SUMMARY OF THE DISCLOSURE

In an embodiment, an implant assembly comprising: niobium (Nb); and titanium (Ti), wherein the implant assembly is configured to expand a first predetermined amount along substantially a first plane at a first temperature and to expand a second predetermined amount along substantially a second plane at a second temperature, wherein the first plane is substantially perpendicular to the second plane, and wherein the second temperature is higher than the first temperature.

In an alternate embodiment, an implant assembly comprising: a first portion comprising a first shape memory alloy; and a second portion comprising a second shape memory alloy; where in each of the first shape memory alloy and the second shape memory alloy comprise nickel (Ni) and titanium (Ti); wherein the first shape memory alloy is configured to expand a first predetermined amount along substantially a first plane at a first temperature, wherein the second shape memory alloy is configured to expand a second predetermined amount along substantially a second plane at a second temperature; wherein the first plane and the second plane are substantially perpendicular, and wherein the second temperature is higher than the first temperature.

In an embodiment, a method of making a shape memory alloy implant assembly comprising: melting and homogenizing titanium (Ti) and niobium (Nb) to form a first shape memory alloy and a second shape memory alloy; forming a first portion of an implant assembly from the first shape memory alloy; forming a second portion of the implant assembly from the second shape memory alloy; shape-setting the first and the second shape memory alloys, wherein, subsequent to the shape-setting the first shape memory alloy is configured to expand a first predetermined amount along substantially a first plane at a first temperature and the second shape memory alloy is configured to expand a second predetermined amount along substantially a second plane at a second temperature, wherein the first plane and the second plane are substantially perpendicular, and wherein the second temperature is higher than the first temperature.

The present disclosure relates to shape memory alloy implants and methods of making the same, such as shape memory alloy implants for the spine, knee, hip, shoulder, elbow facial structures, or other musculoskeletal structures. The present disclosure further relates to shape memory alloy spinal implants and methods of making the same. The shape memory alloy implants are biocompatible and exhibit an ultra-low effective elastic modulus.

Elastic modulus refers to the tendency of a material to deform in response to the application of force. Elastic modulus increases as the rigidity, or stiffness, of the material increases. The shape memory alloys disclosed herein, can be used, for example, in medical applications, partially implanted devices, external fixation devices, dental implants, and implantable prostheses. Materials that are used to construct implants must possess numerous characteristics to be viable for use. Such characteristics include, but are not limited to, high durability over the lifetime of the patient, high corrosion resistance, shape and size retention, biocompatibility, non-toxicity, prevention of reduction of surrounding bone mass (resorption) and bone quality, and effective carrying and balancing of load.

With respect to load carrying and the prevention of loss of surrounding bone mass and quality, bone resorption caused by stress shielding in patients receiving implants is frequently observed. In many cases, bone adjacent or connected to an implant can suffer from a reduction in density of up to 50% within the first three years after surgery. Overall, 33% of patients may exhibit reduction in density 5-13 years after surgery.

The initial fabrication and processing of shape memory alloys for use in medical implants, which can comprise titanium (Ti) along with one or more of niobium (Nb), nickel (Ni) or zirconium (Zr), as well as oxygen (O) and nitrogen (N), can produce alloys that can be trained to exhibit self-adaptive behavior and an ultra-low effective modulus. Training is defined as the cyclic deformation (loading-unloading) of the alloy at a constant temperature that causes a reduction in its transformation stress (ε_{SIM}). A tailored training process may be a process designed to target a range of percent expansion relative to a component or between a first component and a second component, where each component is tailored to expand within a percentage of the other, or wherein there is a ratio of expansion between at least a first and a second component.

With respect to shape memory alloys comprising Ti and Nb, an ultra-low modulus of elasticity is defined as less than 30 GPa. With respect to shape memory alloys comprising Ti and Ni, an ultra-low modulus of elasticity is defined as less than 40 GPa. For purposes of this disclosure, the modulus of elasticity is referred to as the effective modulus. In order to be trained, the alloy should preferably 1) show superelasticity at a temperature equal to or greater than the desired operational temperature if the material (for example, the temperature range of a human body), 2) the cyclic superelastic deformation of the alloy should preferably allow for a reduction in transformation stress (ε_{SIM}), which is defined as the stress where the stress-strain curve is no longer a straight line, and 3) the cyclic superelastic deformation of the precursor should preferably allow for a reduction in the irrecoverable strain (ε_{IRR}) every cycle. ε_{IRR} should be zero where the minimum ε_{SIM} is observed. In addition, (ε_{SIM}), should be as low as possible. The specific treatment(s) required for a precursor alloy to obtain the above qualities depends of the type and composition of the alloy used. As discussed further herein, the alloys are heat-treated, which can be by solution-treatment, prior to thermo-mechanical processing and training. The initial heat-treatment and subsequent thermo-mechanical processing can comprise a plurality of processing steps which depend on the alloy, the composition, and the desired end use.

While specific processing routes can be determined by alloy, composition, and end use, the training process can be generally described as follows. First, training should be performed under load conditions that are similar to those expected during use. For example, if the material is expected to carry tensile load during use, training should be performed under tension. Second, training should be performed to a strain level greater than the expected maximum strain from use. For example, if an alloy is expected to deform to a maximum of 3% strain in use, the alloy should be trained to a strain exceeding 3%. Third, a higher training strain results in a faster reduction in σ_{SIM}. Fourth, the maximum training strain should not exceed maximum superelastic strain by more than 1-2%. Fifth, training cycles should be carried out until the minimum σ_{SIM} is obtained, or a desired level is reached. Finally, the training can be carried out at a temperature slightly higher than the expected operating temperature of the alloy.

The alloys described herein are self-adaptive, biocompatible, corrosion resistant, shape memory alloys. Self-adaptive is a term used to describe the response of a material to changing conditions, and is a property that may be desired in products that undergo cyclic strain, or other parts where these characteristics are desirable, such as orthopedic or dental implants or mechanical components for engines, turbines, electronic devices, communications systems, and other suitable applications for the purpose of stress-shielding. With respect to orthopedic and dental implants, stress shielding may occur because the implant carries a larger portion of the load than the surrounding bone due to the higher elastic modulus of the implant. For implants of conventional metals or metal alloys, the effect of stress will naturally intensify as bone resorption and loss leads to further transfer of load to the implant, accelerating bone loss. As the surrounding bone gets weaker, the implant will carry a greater load. If the implant can put more weight on the bone, however, the bone is less likely to become compromised. Ti—Nb and Ti—Ni shape memory alloys exhibit cyclic softening and, if bone loss occurs that leads to increased load to the implant, the effective modulus of the implant will be reduced and return the load back to the surrounding bone, preventing further degradation and promoting bone growth and healing. The stress-dependence of the effective modulus allows the implant to adjust its properties based on the operating environment in that if a load carried by the implant is higher than desired, the implant will experience a reduction in effective modulus to transfer load back to surrounding bone. The self-adaptive behavior of such alloy systems may therefore counteract or slow the progression of bone loss by promoting bone growth and healing.

The properties of the shape memory alloys described herein are facilitated by a solid-to-solid phase transformation between Martensite and Austenite crystal structures. This phase transformation occurs through a coordinated shear movement of atoms over very short distances (on the order of Angstroms) where the atoms retain their neighboring relationship with one another. The high temperature austenite phase transforms to a low temperature martensite phase upon cooling. The atomic crystal structure change that occurs during phase transformation is accompanied by a macroscopic change of the alloy that alters the size and shape of the alloy.

In the absence of stress, austenite transforms to a twinned martensite crystal structure upon cooling to accommodate strain caused by the change in crystal structure. Twinned martensite is composed of multiple twin-related lattice correspondence variants. When stress is applied, the martensite may detwin, resulting in a single lattice correspondence structure and net shape change. When martensite forms inside austenite, the differing crystal structures produce regions of local strain. This strain is too large for accommodation in a purely elastic fashion. Instead, in shape memory alloys, the strain is accommodated by producing a twinned martensite structure. When the higher symmetry austenite transforms to the lower symmetry martensite, it may do so via several martensite lattice corresponding variants. The number of such variants that can be formed is determined by the martensite and austenite crystal structures. By forming a twinned structure variant, the martensite is able to accommodate a large portion of strain due to crystal structure transformation. Under an external biasing stress, certain variants become energetically favored over others, leading to a martensite reorientation. Martensite may also detwin, leading to other energetically favored lattice variants. Both martensite reorientation and detwinning result in macroscopic size and shape change, and give rise to shape memory behavior and superelasticity. The martensitic transformation can be thermally or stress-induced and conforms to the Clausius-Claperyon relationship.

The deformation response of shape memory alloys depends on the testing temperature relative to the transformation temperatures martensitic start (Ms), martensitic finish (Mf), austenitic start (As), and austenitic finish (Af) of the alloy. If the material is deformed below the Mf temperature in a self-accommodating martensite structure, then the strain is accommodated by the growth of one variant favored by the stress at the expense of others, as well as detwinning. As all martensite variants are equally stable in the absence of external of internal stresses, the martensite structure stays in the reoriented and detwinned state, and remains in the deformed shape after loading. When heated above Af after unloading, all martensite transforms to austenite. When austenite is cooled below Mf, the martensite will again form in a self-accommodating state, and all deformations from detwinning are recovered in the absence of plasticity in what is known as a one-way shape memory effect. Here, the austenite shape of the alloy is “remembered”, and the material can return to this shape even after deformation to the martensite state. This ability enables the fabrication of deployable biocompatible implants that are deployed in a deformed martensite state, exhibiting a shrunken size and shape, and then transition into the desired size and shape as it is warmed to body temperature. This transition facilitates implantation in the patient and reduces the possibility of detrimental effects to the patients during and after surgery and rehabilitation.

If sufficient stress is applied in the austenite state, the austenite may transform into martensite in a single-variant configuration, which results in a macroscopic size and shape change. When the stress is removed, however, the martensite structure becomes unstable and reverts back to austenite, and recovers this change in size and shape. This effect is known as superelasticity. The material is able to sustain a large amount of recoverable strain, from 5-25%, depending on the alloy and composition.

As shape memory alloys experience multiple forward-reverse transformation cycles, their shape memory and superelastic properties undergo changes caused by defects that are generated and modified by the transformation. These changes may include an increase or decrease in transformation temperatures, reduction of stress, reduction of thermal hysteresis, accumulation of irrecoverable strain, and changes in the hardening rate of the stress-strain curve. Traditionally, cyclic functional fatigue may be undesirable since it alters the properties of the alloy, causing it to deviate from its desired engineering response. However, functional fatigue can generally be stabilized after a number of transformation cycles, such that further cycles no longer affect properties appreciably. Shape memory alloy components are subjected to such a stabilization process known as training to improve alloy response predictability.

These transformation induced changes are caused either by the generation of defects or retained martensite during transformation. As interfaces between martensite and austenite form and move, defects are generated at the interfaces of the two structures, causing dislocations to form to relieve accumulated internal stress. The precise effect of cyclic functional fatigue depends on the method of transformation. When a shape memory alloy transforms from changes in temperature under stress-free conditions, the transformation temperatures generally decrease. As transformation occurs, dislocations at interfaces between martensite and austenite. However, since no stress is applied, martensite is formed in the self-accommodated state and the stress field from the generated defects is randomly oriented. This means that no specific martensite variant is favored by the defects and, as a result, the defects hinder phase transformation by acting as obstacles to the movement of the phase front in subsequent transformation cycles. Austenite is stabilized over the martensite and transformation temperatures are decreased.

If thermally-induced transformation occurs under applied stress, or if transformation is stress-induced, transformation cycles will increase the transformation temperatures of the alloy. Dislocations formed during these cycles are oriented and favor specific martensite variants, and martensite becomes stabilized compared to austenite. As discussed above, an important consideration in choosing an appropriate shape memory alloy for medical implants is their cyclic response to stress-induced phase transformation. Stress-induced phase transformation cycles cause three main changes in the stress-strain response of the alloy: 1) transformation stress will gradually decrease with increasing number of cycles due to increased transformation temperature; 2) transformation stress hysteresis will be reduced; and 3) irrecoverable strain will be accumulated.

The aforementioned changes in superelastic properties due to low-cycle functional fatigue have largely been explained by the generation of dislocations at the phase interfaces, similar to the explanation used for thermally-induced transformation of shape memory alloys. However, retained martensite may play a role in changes in superelastic behavior. When the material is loaded again, it no longer requires nucleation of martensite since martensite already exists in the matrix. Instead, martensite simply grows. Since nucleation is reduced or eliminated, the energy barrier for the austenite to martensite transformation is significantly reduced, thus the stress required to trigger stress-induced phase transformation is also reduced. The martensite retained by dislocations, but other mechanisms, such as point defects and precipitates, have also been suggested as possibilities.

The present disclosure relates to shape memory alloy implants that exhibit the properties described above and methods of making the same. Such shape memory alloy implants for the spine, knee, hip, shoulder, elbow, facial structures, or other musculoskeletal structures are envisioned. The present disclosure further relates to shape memory alloy spinal implants and methods of making the same. The shape memory alloy implants exhibit an ultra-low effective elastic modulus and are biocompatible.

In one embodiment, disclosed herein is a spinal implant comprises a shape memory alloy. The shape memory alloy comprises niobium (Nb) and titanium (Ti), wherein substantially pure Nb and Ti are melted and homogenized to form an alloy, and wherein the spinal implant is formed by a shape-setting process that comprises at least one of a thermo-mechanical process, a machining process and a training process where cyclic loads are applied. In some embodiments, the shape-setting process may be performed in one step, and in other embodiments it may be performed in a series of steps comprising one or more cycles or the thermo-mechanical process, the machining process and the training process.

In another embodiment, disclosed herein is a spinal implant comprises a shape memory alloy. The shape memory alloy comprises Nb, Ti and zirconium (Zr), wherein substantially pure Nb, Ti and Zr are melted and homogenized to form an alloy, and wherein the spinal implant is formed by a shape-setting process that comprises at least one of a thermo-mechanical process, a machining process and a training process training process.

In yet another embodiment, disclosed herein is a spinal implant comprising a shape memory alloy. The shape memory alloy comprises Nb, Ti and oxygen (O) wherein substantially pure Nb and Ti are melted and homogenized with titanium dioxide (Ti0₂) to form an alloy, and wherein the spinal implant is formed by a shape-setting process that comprises at least one of a thermo-mechanical process, a machining process and a training process.

In yet another embodiment, disclosed herein is a spinal implant comprising a shape memory alloy. The shape memory alloy comprises Nb, Ti and nitrogen (N) wherein substantially pure Nb and Ti are melted and homogenized with titanium nitride (TiN) to form an alloy, and wherein the spinal implant is formed by a shape-setting process that comprises at least one of a thermo-mechanical process, a machining process and a training process.

In yet another embodiment, disclosed herein is a spinal implant comprising a shape memory alloy. The shape memory alloy comprises nickel (Ni) and titanium (Ti), wherein substantially pure Ni and Ti are melted and homogenized to form an alloy, and wherein the spinal implant is formed by a shape-setting process that comprises at least one of a thermo-mechanical process, a machining process and a training process.

In one embodiment, disclosed herein is a spinal implant comprising more than one shape memory alloy such that different regions of the implant, corresponding to different shape memory alloys, undergo shape memory changes over different respective temperature ranges. The implant can be fabricated such that, for example, a first portion of the implant, corresponding to a first shape memory alloy, can expand over a first temperature range in a substantially perpendicular direction relative to the spinal column and a second portion of the implant, corresponding to a second shape memory alloy, can expand over a second temperature range higher than the first temperature range in a substantially parallel direction relative to the spinal column. Here, the first and second shape memory alloys can be composed of the same chemical elements, for example Ti:Nb, but each having a different composition. The first and second shape memory alloys can alternatively be composed of different elements (e.g., different base alloys), for example Ti:Ni and Ti:Nb. In some embodiments, the first portion may also expand in the second direction at the first temperature and the second portion may also expand in the first direction at the second temperature.

In another exemplary aspect, methods for making a shape memory alloy spinal implant are also provided. In general, the method comprises melting and homogenizing substantially pure titanium (Ti) and niobium (Nb) to form the alloy, machining the alloy into the shape of a spinal implant, and, in some embodiments, further modifying the shape of the implant or training the spinal implant to tailor the transformation temperature. In at least one example, the method can further include a thermo-mechanical processing procedure including hot extrusion, warm extrusion, cold extrusion, rolling, swaging, equal channel angular pressing, equal channel angular extrusion and wire drawing, or combinations thereof, prior to machining. The thermo-mechanical processing procedure can be used to increase the strength and stability of the alloy.

In yet another exemplary aspect, methods for making a shape memory alloy spinal implant are also provided. In general, the method comprises melting and homogenizing substantially pure titanium (Ti), niobium (Nb) and zirconium (Zr) to form the alloy, machining the alloy into the shape of a spinal implant, and training the spinal implant. In at least one example, the method can further include a thermo-mechanical processing procedure including hot extrusion, warm extrusion, cold extrusion, rolling, swaging, equal channel angular pressing, equal channel angular extrusion and wire drawing, or combinations thereof, prior to machining. The thermo-mechanical processing procedure can be used to increase the strength and stability of the alloy.

In yet another exemplary aspect, methods for making a shape memory alloy spinal implant are also provided. In general, the method comprises melting and homogenizing substantially pure titanium (Ti) and niobium (Nb) with titanium dioxide (Ti0₂) to form the alloy, machining the alloy into the shape of a spinal implant, and training the spinal implant. In at least one example, the method can further include a thermo-mechanical processing procedure including hot extrusion, warm extrusion, cold extrusion, rolling, swaging, equal channel angular pressing, equal channel angular extrusion and wire drawing, or combinations thereof, prior to machining. The thermo-mechanical processing procedure can be used to increase the strength and stability of the alloy.

In yet another exemplary aspect, methods for making a shape memory alloy spinal implant are also provided. In general, the method comprises melting and homogenizing substantially pure titanium (Ti) and niobium (Nb) with titanium nitride (TiN) to form the alloy, machining the alloy into the shape of a spinal implant, and training the spinal implant. In at least one example, the method can further include a thermo-mechanical processing procedure including hot extrusion, warm extrusion, cold extrusion, rolling, swaging, equal channel angular pressing, equal channel angular extrusion and wire drawing, or combinations thereof, prior to machining. The thermo-mechanical processing procedure can be used to increase the strength and stability of the alloy.

In yet another exemplary aspect, methods for making a shape memory alloy spinal implant are also provided. In general, the method comprises melting and homogenizing substantially pure titanium (Ti) and nickel (Ni) to form the alloy, machining the alloy into the shape of a spinal implant, and training the spinal implant. In at least one example, the method can further include a thermo-mechanical processing procedure including hot extrusion, warm extrusion, cold extrusion, rolling, swaging, equal channel angular pressing, equal channel angular extrusion and wire drawing, or combinations thereof, prior to machining. The thermo-mechanical processing procedure can be used to increase the strength and stability of the alloy.

In another exemplary aspect, methods for making a shape memory alloy spinal implant are also provided. In general, the method comprises forming more than one shape memory alloy wherein each alloy has a different composition and temperature dependent shape memory characteristics, connecting the more than one shape memory alloys together, forming the connected alloys into a shape of a spinal implant, and training the spinal implant. The more than one shape memory alloy can be directly connected by, for example, laser welding. The more than one shape memory alloys can also be indirectly connected by fusing each to another material such as, for example, a plate or strip of pure titanium or with one or more biocompatible adhesives. In one example, disclosed herein is a spinal implant comprising more than one shape memory alloy where different regions of the implant, corresponding to different shape memory alloys, undergo shape memory changes over different temperature ranges. The implant can be fabricated such that, for example, a first portion of the implant, corresponding to a first shape memory alloy, can expand over a first temperature range in a substantially perpendicular direction relative to the spinal column and a second portion of the implant, corresponding to a second shape memory alloy, can expand over a second temperature range higher than the first temperature range in a substantially parallel direction relative to the spinal column. Here, the first and second shape memory alloy can be composed of the same chemical elements, for example Ti:Nb, but each having a different composition. The first and second shape memory alloys can alternatively be composed of different elements, for example Ti:Ni and Ti:Nb. In at least one example, the method can further include a thermo-mechanical processing procedure including hot extrusion, warm extrusion, cold extrusion, rolling, swaging, equal channel angular pressing, equal channel angular extrusion and wire drawing, or combinations thereof, prior to forming of the spinal implant. The thermo-mechanical processing procedure can be used to increase the strength and stability of the alloy.

In another exemplary aspect, processes for implanting a spinal implant are also provided. In general, the spinal implant may be inserted into a vertebral cavity corresponding to the removed compromised disc. The spinal implant is kept at a low temperature relative to room temperature, exhibits a shrunken first size and shape and is introduced into a vertebral cavity of a patient. Upon introduction of the spinal implant, the implant will expand to a larger, predetermined, second size and shape in response to the body temperature of the patient.

In one embodiment, disclosed herein is a spinal implant comprising a shape memory alloy. The shape memory alloy comprises niobium (Nb) and titanium (Ti), wherein substantially pure Nb and Ti are melted and homogenized to form an alloy, and wherein the spinal implant is formed by at least one of a thermo-mechanical process, a machining process and a training process.

The relative amount of Nb and Ti, in atomic percent (at %), in a shape memory alloy spinal implant is between 20-30% Nb and between 70-80% Ti respectively, preferably between 23-27% Nb and between 73-77% Ti respectively, and more preferably between 24-26% Nb and between 74-76% Ti respectively. It will be understood by those skilled in the art that suitable ranges include ranges include any combination of the specified atomic percentages even if the specific combination is not listed herewith.

In another embodiment, disclosed herein is a spinal implant comprising a shape memory alloy. The shape memory alloy comprises Nb, Ti and zirconium (Zr), wherein substantially pure Nb, Ti and Zr are melted and homogenized to form an alloy, and wherein the spinal implant is formed by at least one of a thermo-mechanical process, a machining process and a training process.

The relative amount of Nb, Ti and Zr, in at %, in a shape memory alloy spinal implant is between 18-26% Nb, between 70-80% Ti and between 4-12% Zr respectively, and preferably between 20-24% Nb, between 70-80% Ti and between 6-10% Zr respectively. It will be understood by those skilled in the art that suitable ranges include ranges include any combination of the specified atomic percentages even if the specific combination is not listed herewith.

In yet another embodiment, disclosed herein is a spinal implant comprising a shape memory alloy. The shape memory alloy comprises Nb, Ti and oxygen (O) wherein substantially pure Nb and Ti are melted and homogenized with titanium dioxide (Ti0₂) to form an alloy, and wherein the spinal implant is formed by a shape-setting process that comprises at least one of a thermo-mechanical process, a machining process, and a cyclic-loading training process.

The relative amount of Nb and Ti and O, in atomic percent (at %), in a shape memory alloy spinal implant is between 20-30% Nb, between 70-80% Ti and between 0.1-2% O respectively, preferably between 20-30% Nb, between 70-80% Ti and between 0.5-1.5% O respectively, and more preferably between 20-30% Nb, between 70-80% Ti and about 1% O respectively. It will be understood by those skilled in the art that suitable ranges include ranges include any combination of the specified atomic percentages even if the specific combination is not listed herewith.

In yet another embodiment, disclosed herein is a spinal implant comprising a shape memory alloy. The shape memory alloy comprises Nb, Ti and nitrogen (N) wherein substantially pure Nb and Ti are melted and homogenized with titanium nitride (TiN) to form an alloy, and wherein the spinal implant is formed by a shape-setting process that comprises at least one of a thermo-mechanical process, a machining process, and a training process.

The relative amount of Nb and Ti and N, in atomic percent (at %), in a shape memory alloy spinal implant is between 20-30% Nb, between 70-80% Ti and between 0.1-2% N respectively, preferably between 20-30% Nb, between 70-80% Ti and between 0.5-1.5% N respectively, and more preferably between 20-30% Nb, between 70-80% Ti and about 1% N respectively. It will be understood by those skilled in the art that suitable ranges include ranges include any combination of the specified atomic percentages even if the specific combination is not listed herewith.

In yet another embodiment, disclosed herein is a spinal implant comprising a shape memory alloy. The shape memory alloy comprises nickel (Ni) and titanium (Ti), wherein substantially pure Ni and Ti are melted and homogenized to form an alloy, and wherein the spinal implant is formed by a shape-setting process that comprises at least one of a thermo-mechanical process, a machining process, and a training process.

The relative amount of Ni and Ti, in at %, in a shape memory alloy spinal implant is between 45-52% Ni and between 48-55% Ti respectively, preferably between 48-51% Ni and between 49-52% Ti respectively, and more preferably between 50.1-50.9% Ni and between 49.1-49.9% Ti respectively. It will be understood by those skilled in the art that suitable ranges include ranges include any combination of the specified atomic percentages even if the specific combination is not listed herewith.

In alternate embodiments, any of the shape memory alloy compositions disclosed above may also further comprise aluminum (Al), chromium (Cr), cobalt (Co) gallium (Ga), germanium (Ge), gold (Au), molybdenum (Mo), palladium (Pd), platinum (Pt), scandium (Sc), silicon (Si), silver (Ag), tantalum (Ta), tin (Sb), vanadium (V), or any other biologically suitable metal.

In alternate embodiments, disclosed herein is a spinal implant comprising more than one shape memory alloy such that different regions of the implant, corresponding to different shape memory alloys, undergo shape memory changes over different temperature ranges. The implant can be fabricated such that, for example, a first portion of the implant, corresponding to a first shape memory alloy, can expand over a first temperature range in a substantially perpendicular direction relative to the spinal column and a second portion of the implant, corresponding to a second shape memory alloy, can expand over a second temperature range higher than the first temperature range in a substantially parallel direction relative to the spinal column. Here, the first and second shape memory alloys can be composed of the same chemical elements, for example Ti:Nb, but each having a different composition such as for example, a first alloy comprising Ti:Nb with an at % of 76:24 and a second alloy comprising Ti:Nb with an at % of 75:25. The first and second shape memory alloys can alternatively be composed of different element combinations, for example Ti:Ni and Ti:Nb. It will be understood by those skilled in the art that suitable ranges include ranges include any combination of the specified atomic percentages even if the specific combination is not listed herewith. It will also be understood by those skilled in the art that any of the above described shape memory alloys can be used to form a spinal implant comprising more than one shape memory alloy.

In at least one embodiment, the spinal implant is substantially circular in shape along an axis perpendicular to the spinal column. In alternate embodiments, the spinal implant can substantially take the form of a rectangle, square, ellipse, diamond, oval, D-shape, U-shape or any shape desired to conform and substantially match the structure of a compromised disc being replaced and to optimize interaction of the spinal implant with adjacent vertebrae. In at least one embodiment, the spinal implant can be substantially cylindrical with a substantially uniform diameter along an axis parallel with the spinal column. In alternative embodiments, the spinal implant can have a diameter which decreases from the top and bottom surfaces of the spinal implant toward a center of the spinal implant. In further alternative embodiments, the spinal implant can have a pivotable member, made of the same or similar shape memory alloy or other suitable material, connected to and interposed between the top and bottom surfaces of the spinal implant. In further alternate embodiments, the spinal implant can have a compressible member connected to and interposed between the top and bottom surfaces of the spinal implant. The outer surface of the disc implant can be roughened or corrugated, or have a plurality of recesses or apertures running therethrough. It will be understood by those skilled in the art that the above described shape memory alloys can be fabricated into any suitable shape and size and the shapes described herein are not intended to limit the scope of this disclosure.

An implant assembly comprising a shape memory alloy, the shape memory alloy comprising: niobium (Nb); and titanium (Ti), wherein the implant assembly is configured to be a first shape and size at a first temperature and wherein, in response expand to a second shape and size at a second temperature, and wherein the second temperature is higher than the first temperature, wherein the implant assembly is a spinal implant assembly, wherein the implant assembly is configured to expand along substantially a first plane at a first temperature and to expand along substantially a second plane at a second temperature, wherein the first plane and the second plane are substantially perpendicular, and wherein the second temperature is higher than the first temperature, wherein the expansion of the first plane and the second plane are configured to result in an expanded implant assembly configured to conformance fit to a cavity of a patient, wherein the shape memory alloy comprises an atomic % of Nb between 20-30% and an atomic % of Ti between 70-80%, wherein the shape memory alloy further comprises zirconium (Zr), wherein the shape memory alloy comprises an atomic % of Nb between 18-26%, an atomic percentage of Ti between 70-80%, and an atomic % of Zr between 4-12%. The embodiment further comprising wherein the shape memory alloy further comprises an atomic % of oxygen (O) between 0.1-2%, wherein the shape memory alloy further comprises an atomic % of nitrogen (N) between 0.1-2%, and, wherein the implant assembly comprises two or more shape memory alloy compositions. As discussed herein, an implant assembly may comprise two or more portions which may also be described as components, these components may differ in either or both of the respective alloy compositions and/or the shape-setting process employed. The compositions may differ by the concentration of a single element or multiple elements, and one composition may comprise elements that may not be present in the other composition. The shape-setting processes may differ by type of process and/or by the number of cycles employed, and/or by the process parameters employed during shape-setting. In some embodiments, only the machining and thermos-mechanical processing may be performed. The training comprising cyclic loading may be performed when a lower elastic modulus/stiffness is desired for a particular application. In an alternate embodiment, an implant assembly comprising a shape memory alloy, the shape memory alloy comprising: nickel (Ni); and titanium (Ti), wherein the implant assembly is configured to be a first shape and size at a first temperature and expand to a second shape and size at a second temperature, and wherein the second temperature is higher than the first temperature, wherein the implant assembly is a spinal implant assembly, wherein the implant assembly is configured to expand along substantially a first plane at a first temperature and to expand along substantially a second plane at a second temperature, wherein the first plane and the second plane are substantially perpendicular, and wherein the second temperature is higher than the second temperature, wherein the expansion of the first plane and the second plane is configured to result in an expanded implant assembly configured to conformance fit to a cavity of a patient, wherein the shape memory alloy comprises an atomic % of Ni between 48-55% and an atomic % of Ti between 45-52%, wherein the implant assembly comprises two or more shape memory alloy compositions.

In embodiment, a method of making a shape memory alloy implant assembly comprising: melting and homogenizing titanium (Ti) and niobium (Nb) to form the alloy; machining the alloy into a shape of an implant; and training the alloy such that the transformation temperature is tailored so that implant assembly exhibits a first predetermined size and shape at a first temperature and expands to a second predetermined size and shape at a second temperature, wherein the second temperature is higher than the first temperature, thermo-mechanically processing the alloy prior to machining, wherein the alloy is machined into the shape of a spinal implant assembly, wherein the formed shape memory alloy comprises an atomic % of Nb between 20-30% and an atomic % of Ti between 70-80%, wherein zirconium (Zr) is melted and homogenized with Ti and Nb to form the alloy. In an alternate embodiment, the formed shape memory alloy comprises an atomic % of Nb between 18-26%, an atomic % of Ti between 70-80%, and an atomic % of Zr between 4-12%, wherein titanium oxide (Ti0₂) is melted and homogenized with Ti and Nb to form the alloy, wherein the formed shape memory alloy further comprises an atomic % of oxygen (O) between 0.1-2%, wherein titanium nitride (TiN) is melted and homogenized with Ti and Nb to form the alloy, and wherein the formed shape memory alloy further comprises an atomic % of nitrogen (N) between 0.1-2%. The embodiment further comprising wherein thermo-mechanical processing comprises hot, warm, or cold deformation, wherein deformation includes pressing, extrusion, rolling, swaging, equal channel angular extrusion and wire drawing, or combinations thereof, wherein the thermo-mechanical processing comprises holding the alloy between 150° C.-800° C., wherein the thermo-mechanical processing lasts for, 1 minute to one hour, or from 1 hr.-24 hrs., or from 24 hrs.-72 hrs., wherein the thermo-mechanical processing is followed by a water quench. The method further comprising, wherein the thermo-mechanical processing comprises more than 10% of Von mises equivalent strain. In an embodiment, subsequent after machining, the alloy undergoes a heat treatment process, wherein the heat treatment process comprises 300° C. to 600° C. treatments from 30 seconds to 10 minutes followed by a water quench or cooling to room temperature, wherein the training comprises cyclic loading on the alloy to strains larger than 0.5% strain in the austenite phase at a temperature below 150° C. In an embodiment, training comprises a cyclic loading on the alloy to 1-3% strain at a temperature between 20° C. (68° F.)-50° C. (122° F.), wherein the loading and unloading cycle is performed for a plurality of cycles, wherein two or more shape memory alloy compositions are connected prior to machining and the connected shape memory alloy compositions are machined into the shape of an implant assembly and/or wherein two or more shape memory alloy compositions are machined to predetermined shape and connected to form an implant assembly prior to training, wherein each of the two or more shape memory alloy compositions are directly connected/coupled or wherein each of the two or more shape memory alloy compositions are indirectly connected.

In an embodiment, a method of making a shape memory alloy implant assembly comprising: melting and homogenizing titanium (Ti) and nickel (Ni) to form the alloy; machining the alloy into a shape of an implant; and training the alloy such that the implant assembly exhibits a first predetermined size and shape at a first temperature and expands to a second predetermined size and shape at a second temperature, wherein the second temperature is higher than the first temperature, and thermo-mechanically processing the alloy prior to machining, wherein the alloy is machined into the shape of a spinal implant assembly, wherein the formed shape memory alloy comprises an atomic % of Ni between 48-55% and an atomic % of Ti between 45-52%, wherein thermo-mechanical processing comprises hot, warm, or cold deformation, wherein deformation includes pressing, extrusion, rolling, swaging, equal channel angular extrusion and wire drawing, or combinations thereof. In an embodiment, the thermo-mechanical processing holding the alloy between 150° C.-800° C. and is performed from one minute to one hour or from 1 hr.-24 hrs. or from 24 hrs.-72 and is followed by a water quench or cooling to room temperature. The embodiment further comprising wherein the cold-working process comprises more than 10% of Von mises equivalent strain or wherein the hot-working process comprises more than 50% of Von mises equivalent strain, wherein, subsequent to machining, the alloy undergoes a heat treatment process, wherein the heat treatment process comprises holding the alloy at 200° C. to 500° C. for 15 minutes to 100 hours, wherein the required treatment time is reduced as the treatment temperature is increased. In an embodiment, the training a comprises cyclic loading on the alloy to 1-5% strain at a temperature between 20° C. (68° F.)-50° C. (122° F.), wherein the training comprises cyclic loading on the alloy to strains larger than 0.5% strain in the austenite phase at a temperature below 150° C., wherein two or more shape memory alloy compositions are connected prior to machining and the connected shape memory alloy compositions are machined into the shape of an implant assembly, wherein each of the two or more shape memory alloy compositions are machined to predetermined shape and connected to form an implant assembly prior to training, wherein each of the two or more shape memory alloy compositions are directly connected or indirectly connected. The two or more shape memory compositions used in an implant assembly may comprise different alloys comprising different elements, or may comprise different alloys comprising some overlapping elements and some different elements at varying concentrations.

Embodiments described herein comprise a combination of features and advantages intended to address various shortcomings associated with certain prior devices, systems, and methods. The foregoing has outlined rather broadly the features and technical advantages of the invention in order that the detailed description of the invention that follows may be better understood. The various characteristics described above, as well as other features, will be readily apparent to those skilled in the art upon reading the following detailed description, and by referring to the accompanying drawings. It should be appreciated by those skilled in the art that the conception and the specific embodiments disclosed may be readily utilized as a basis for modifying or designing other structures for carrying out the same purposes of the invention. It should also be realized by those skilled in the art that such equivalent constructions do not depart from the spirit and scope of the invention as set forth in the appended claims.

### BRIEF DESCRIPTION OF THE DRAWINGS

For a detailed description of the preferred embodiments of the invention, reference will now be made to the accompanying drawings in which:

FIG. 1 graph of the effects of stress-shielding on bone and self-adaptive features of the shape memory alloys disclosed herein.

FIGS. 2A and 2B are cytotoxicity results from Ti and Ti-based alloys.

FIGS. 3A and 3B are potentiodynamic polarization graphs of corrosion resistance in saline (3A) and modified Hank's solution (3B) environments.

FIG. 4 is a graph of superelastic properties exhibited by Ti—Nb.

FIG. 5 is a graph of multiple stress strain curves resulting from a plurality of training processes.

FIG. 6 is a graph of the stress-strain behavior of various alloys and an inset graph of the effect on the effective modulus of 1, 10, 100, and 1000 cycles.

FIG. 7 is a tensile stress-strain diagram of the A610 alloy before and after 1000 superelastic cycles.

FIGS. 8A-8B are graphs that illustrate the dependence of σ_{SIM} and stress hysteresis on the number of cycles.

FIG. 9 is a graph that illustrates the dependence of transformation stress on the testing temperature.

FIG. 10 is a graph that illustrates how a high number of superelastic cycles at a low strain can be replaced by a lower number of cycles at a higher strain.

FIG. 11 is a flowchart of an embodiment of a method of manufacturing an ultra-low effective elastic modulus with shape memory properties.

FIG. 12 is a flowchart of an alternate embodiment of a method of manufacturing a self-adaptive shape-memory alloy with an ultra-low effective modulus of elasticity.

FIG. 13 is an isometric view of an exemplary embodiment of a shape memory alloy spinal implant in a shrunken state.

FIG. 14 is a bottom plan view of the shape memory alloy spinal implant of FIG. 13 in the shrunken state.

FIG. 15 is an isometric view of the shape memory alloy spinal implant of FIG. 13 in an expanded state;

FIG. 16 is a bottom plan view of the shape memory alloy spinal implant of FIG. 13 in the expanded state; and

FIG. 17 is an isometric view of another exemplary embodiment of a shape memory alloy spinal implant.

### DETAILED DESCRIPTION OF THE PREFERRED EMBODIMENTS

The following discussion is directed to various exemplary embodiments. However, one skilled in the art will understand that the examples disclosed herein have broad application, and that the discussion of any embodiment is meant only to be exemplary of that embodiment, and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

Several definitions that apply throughout this disclosure will now be presented.

Throughout this disclosure, atomic percentage, or at %, is defined as the percentage of one kind of atom relative to the total number of atoms.

The term “substantially” is defined to be essentially conforming to the particular dimension, shape or other word that substantially modifies, such that the component need not be exact. For example, substantially cylindrical means that the object resembles a cylinder, but can have one or more deviations from a true cylinder.

Where used, the term “comprising,” means “including, but not necessarily limited to”; it specifically indicates opening-ended inclusion or membership in the so-described combination, group, series and the like.

In the following disclosure the term “coupled” is defined as connected, whether directly or indirectly through intervening components, and is not necessarily limited to physical connections. The connection can be such that the objects are permanently connected or releasably connected.

In one or more exemplary embodiments there is disclosed herein a shape memory alloy spinal implant, and method for making a spinal implant, and method for implantation of an spinal implant. Disclosed herein also are multiple exemplary embodiments involving replacement of intervertebral discs employing minimally invasive techniques. As described herein, the spinal implants are preferably dynamically integrated to provide for the maintenance of natural anatomy and balance of forces in the vertebral column.

While the embodiments described herein are for shape memory alloy spinal implants and methods of making the same, other implant structures such as shape memory alloy implants for the knee, hip, shoulder, elbow, facial structures, or other musculoskeletal structures can be formed from the shape memory alloys disclosed herein. Furthermore, the disclosed shape memory alloys can be used in surgical materials such as bone plates or bone rods for fracture fixation, tissue anchors or sutures, couplings for joint replacements, or in fusion procedures across joints such as the sacroiliac joint, wrist, fingers, and feet (for example, hindfoot, midfoot and forefoot).

### Mechanical Properties of SMAs

Disclosed herein is a method of fabricating shape-memory alloys that have an ultra-low effective elastic modulus and are biocompatible. The elastic modulus refers to a material's tendency to deform when a force is applied, a stiffer material will have a higher elastic modulus than a less rigid material. This material may be used, for example, in medical applications, for example, implanted devices, partially implanted devices, external fixation devices, dental implants, and implantable prosthesis. Implants present concerns with respect to the base material and processing of that material because implants not only need to be durable, to reduce loosening, infection, and subsequent revision surgeries, and biocompatible, but also prevent reduction of bone mass (resorption) and bone quality in the area surrounding the implant. The effective elastic modulus of the implant material, often a stainless steel or Co—Cr alloy, is about 10 times higher than the elastic modulus of the human bone. Since load of the body is then carried with the implant and the surrounding bone in parallel, the stiff implant will carry a large majority of the load. The bone, which is a living tissue that constantly adapts itself to the surrounding, will become weaker as a result of lack of loading, according to Wolff's Law. Over time, this leads to the reduction of bone mass (resorption) and bone quality of bones that surrounds the implant. Bone resorption is the term used to describe the process by which bone is broken down by osteoclasts, this releases minerals and transfers calcium to the blood. Bone resorption caused by stress shielding is frequently observed in patients receiving implants. The reduction of mineral density in the bone surrounding an implant may be up to 50% after three years of implantation. Pronounced bone resorption may occur in 33% of patients 5-13 years after receiving the implant. It should be noted that the terms “implant” and “implants” are used interchangeably here because it is understood that the implantation of an orthopedic implant for a knee, hip, spine, elbow, craniomaxillofacial, etc., may actually involve the implantation of the implant itself, as well as rods, screws, and other components that are affixed to or used in conjunction with the implant. Because these components may be implanted, their biocompatibility may be as important as the primary implant, for example, an acetabular cup, femoral implant, hip stem, or tibial implant.

The initial fabrication and processing of material used for implantable devices, which may comprise titanium (Ti) along with niobium (Nb), nickel (Ni), or zirconium (Zr), must produce an alloy that can be trained to show self-adaptive behavior and an ultra-low effective modulus. Training is the cyclic deformation (loading-unloading) of the alloy at a constant temperature that causes a reduction in its transformation stress (σ_{SIM}).

An ultra-low modulus is a modulus of elasticity that is below 30 GPa. For purposes of this disclosure, the modulus of elasticity referred to is the effective modulus. In order to be trained, the precursor material must exhibit certain characteristics including: (1) show super elasticity at a temperature greater or equal to the desired operational temperature of the material, (2) the cyclic superelastic deformation of the precursor must allow for a reduction in the transformation stress (σ_{SIM}) which is defined as the stress where the stress-strain curve is no longer a straight line, and (3) the cyclic superelastic deformation of the precursor must allow for a reduction in the irrecoverable strain (εᵢᵣᵣ) every cycle. This strain should be zero at the cycle where the minimum σ_{SIM} is observed. In addition, the minimum achievable σ_{SIM} should be as low as possible. The specific treatment(s) required in order for a precursor material to obtain the above qualities depends on the type and composition of the alloy used. As discussed below, the alloys are heat-treated, which may be a solution-treatment, prior to thermo-mechanical processing and training. The initial heat treat and the subsequent thermo-mechanical processing may comprise a plurality of processing steps which depend on the alloy, the composition, and the desired end use and operating conditions of that end use. Alloys such as those in the shape-memory alloy families of Ni—Ti, Ti—Nb, Cu—Ni—Al, Cu—Zn—Al, Cu—Mn—Al, Fe—Ni—Co—Al, and Fe—Ni—Mn—Al may meet these requirements.

While the specific processing routes may be determined by alloy, composition, and end use, the training process can still be generalized by several principles. First, training may be performed in a similar load condition as the expected load condition of the material in application. For example, if the material is expected to carry tensile load in application, training should be done in tension. Second, training may be performed to a strain level greater than the expected maximum strain the material is expected to experience in application. For example, if a material is expected to deform to a maximum of 3% strain in application, it should be trained to a strain greater than 3%. Third, a higher training strain results in a faster reduction in σ_{SIM}. Fourth, the maximum training strain should not exceed maximum superelastic strain by about 2%, in some embodiments, the maximum may be about 1%. Fifth, training cycles should be carried out until the minimum σ_{SIM} is obtained, or a desired level of σ_{SIM} has been reached. And finally, the training may be carried out at a temperature slightly higher than the expected operating temperature of the material.

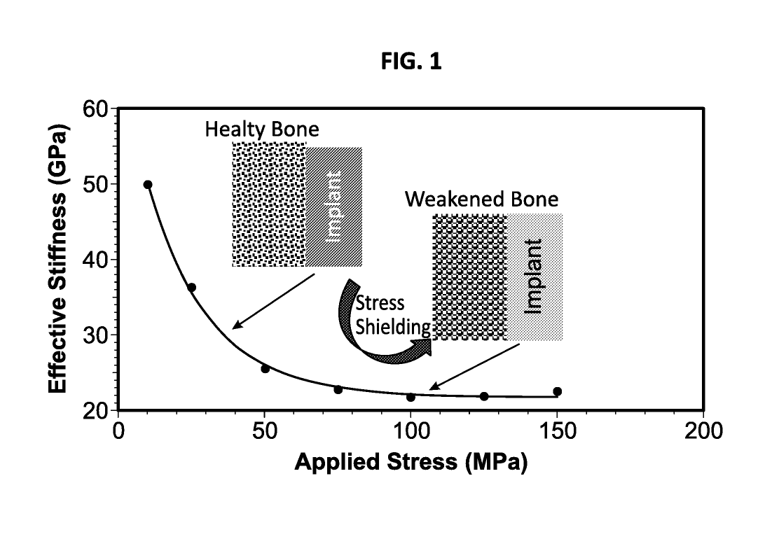

The alloys described herein are self-adaptive, biocompatible, corrosion resistant, shape-memory alloys. Self-adaptive is a term used to describe the response of a material to changing conditions, and this is a property that may be desired in products that undergo cyclic strain, or other parts where these characteristics are desirable, such as orthopedic and dental implants or engine components. The reason that self-adaptive behavior may be desired from an alloy in these cyclic stress situations is because of stress shielding. In the case of orthopedic and dental implants, stress shielding may occur because the implant material carries a larger portion of the load than the surrounding bone, due to the higher elastic modulus of the implant. For implants of conventional metals and alloys, the effect of stress shielding will naturally intensity as bone resorption and loss leads to further transfer of load to the implant, and accelerates the bone loss process. When the bone gets weaker, the implant will carry more of the load. If the implant can put more weight on the bone, the bone is less likely to become weaker. Stress shielding occurs when the bone doesn't see enough load, so if an implant can transfer load to the bone the bone may not weaken as quickly. However, as shown in the cyclically-softened Ti—Nb shape memory alloy in FIG. 1, if bone loss occurs that leads to the transfer of more loads to the implant, the effective elastic modulus of the implant will be reduced and return the load back to the bone, preventing further degradation, and help the bone grown and heal itself. Bone in a healthy person will remodel in response to the loads it is placed under, if a load on a bone decreases, the bone will become less dense and weaker because there is no stimulus for continued modeling that is required to maintain bone mass. The stress-dependence of the effective modulus allows the implant to adjust its properties based on its operating environment: if a higher than desired level of load is carried by the implant, it will automatically reduce its effective modulus to transfer load back to the surrounding. Because the alloy responds, sometimes immediately, to weakening of the bone due to stress shielding by reducing its effective stiffness, the self-adaptive behavior may counteract and slow the progression of bone loss by helping the bone to heal itself.

### Phase Transformation and Mechanical Properties

Martensitic transformation is a solid-to-solid phase transformation that occurs through a coordinated shear movement of atoms over very short (on the order of angstroms) distances where atoms retain their neighboring relationship with one another. The high temperature phase, austenite, transforms to a low temperature phase, martensite, upon cooling. Because the crystal structure of austenite is different than that of martensite, it is possible to obtain a macroscopic shape change that accompanies the transformation.

In the absence of stress, austenite transforms to twined martensite upon cooling in order to accommodate strain caused by a change in crystal structure. The twinned martensite is composed of multiple twin-related lattice correspondence variants. When stress is applied, the martensite may detwin, resulting in a single lattice correspondence variant structure and a net shape change. When martensite forms inside austenite, the difference in their crystal structures generates large local strain. This strain is large enough so it cannot be purely accommodated elastically. Instead in SMAs, the strain is accommodated by producing a twinned martensite structure. When the higher symmetry austenite transforms to the lower symmetry martensite, it may do so in several “ways” called martensite lattice correspondence variants. The number of such variants that can be formed is determined by the crystal structures of the martensite and the austenite. By forming a structure of twin-related lattice correspondence variants, the martensite is able to accommodate a large portion of the strain associated with the change in crystal structure. Under an external biasing stress, certain variants become energetically favored and form or grow at the expense of others in a process known as martensite re-orientation. In addition, the martensite may also detwin, where analogously, the lattice correspondence variant favored under stress grows at the expense of others. Both martensite re-orientation and detwinning results in the macroscopic shape change, and give rise to the shape memory behavior and superelasticity.

The martensitic transformation can be induced both thermally and through the application of stress. In other words, application of stress and reduction in temperature both act as driving forces for the austenite martensite transformation. In fact, there is a linear relationship between the two. This relationship is derived from the thermodynamics relationships of phase transformation and is called the Clausius-Clapeyron relationship. Roughly, it states that

$$\frac{d σ}{dT} = constant .$$

The deformation response of SMAs depends on the testing temperature relative to the transformation temperatures martensitic start (Mₛ), martensitic finish (M_{f}), austenitic start (Aₛ), and austenitic finish (A_{f}), of the alloy. If the material is deformed below the M_{f} temperature in a self-accommodated martensite structure, then the strain is accommodated by the growth of one variant favored by the stress in the expense of others, as well as detwinning. Since all martensite variants are equally stable in the absence of external and internal stresses, the martensite stays in the re-oriented and detwinned state, and remains in the deformed shape after unloading. When heated above A_{f} temperature after unloading, all martensite transforms back to austenite. When the austenite is once again cooled below M_{f}, the martensite will again form in a self-accommodated state, and all deformations from detwinning are recovered in the absence of plasticity; this is the one-way shape memory effect. This means that the austenite shape of the alloy is “remembered”, and the material can return to this shape even after deformation in the martensite state. This ability enables “deployable bio-devices” that are deployed in a deformed martensite state to facilitate easy installation, and then morphs into the desired shape as it is warmed to body temperature.

On the other hand, if sufficient stress is applied in the austenite state, the austenite may transform into martensite in a single-variant configuration, which results in a macroscopic shape change. When the stress is removed, however, the martensite becomes unstable and reverts to austenite, and recovers this change in shape. This effect is known as superelasticity, the material is able to sustain a large amount of recoverable strain, from 5%-25%, depending upon the alloy. The flexibility has been utilized in a number of biomedical applications such as orthodontic arch wires and temporarily bone staples and braces to promote healing.

Conventionally, Ni—Ti SMA (nitinol) is thought to possess much lower elastic modulus than currently used implant alloys. In the austenite state, nitinol is reported to have an elastic modulus of between 60-80 GPa, while in the martensite state, this number is reported to be 30-60 GPa. The reportedly low stiffness would appear to reduce the risks of stress shielding and makes Ni—Ti an attractive implant material: not as a shape memory alloy, but simply a metallic alloy with low elastic modulus. However, the reported low martensite modulus of nitinol is an apparently misleading one. Numerous neutron diffraction, atomistic simulation, and synchrotron diffraction work has shown that the actual elastic modulus of the martensite is well over 100 GPa. The reason that a small modulus is observed in a conventional stress-strain diagram is the simultaneous activation of martensite reorientation and martensite detwinning alongside elastic deformation. Unfortunately, strain caused by martensite reorientation and detwinning does not appear to recover upon unloading, so even at very low applied stress level, irrecoverable strain can be observed in a stress-strain diagram of nitinol in the martensite state. This means that nitinol may not be able to be used in the martensite state as an implant material as it will continuously undergo permanent shape change as stress is applied. However, instead of irreversible mechanisms such as martensite reorientation or detwinning, it may be possible to activate a reversible deformation mechanism, such as stress-induced phase transformation, concurrently with elastic deformation to reduce the effective modulus of the alloy. In most shape memory alloys, including nitinol, the stress required to activate stress-induced phase transformation are quite high (˜200-300 MPa). Since implants do not typically experience such loads, it is apparently not possible to take advantage of this mechanism.

### Ti—Nb Shape Memory Alloys

Ti SMAs with bio-inert constituents such as the Ti—Mo, and the Ti—Nb systems may be suitable for applications such as implants and others where shape-memory properties are needed in conjunction with an ultra-low effective modulus of elasticity. In addition, ternary alloys may be viable with the addition of Ag, Ga, and Sn to the Ti—Mo system, and Al, Ga, Ge. The addition of Pd, Si, Sn, Ta, and Zr to the Ti—Nb alloys may also produce the self-adaptive behavior that may be preferred for these shape-memory alloys. Ti₇₂Nb₂₂Zr₆ currently exhibits the highest total recoverable strain of 4.5% among all Ti alloys other than Ti—Ni. Ti—Nb alloys may be preferred by industries such as the biomedical industry due to high biocompatibility, corrosion resistance, superior workability, and relatively low Young's modulus.

In addition to a reduction of the effective modulus, the mechanical and superelastic properties of the Ti—Nb SMAs must be sufficiently improved through work hardening, grain refinement, and crystallographic texture engineering in order to be considered a feasible material for structural implants. In the experiments in this disclosure, severe plastic deformation technique Equal Channel Angular Extrusion (ECAE) and training were used to achieve the lowered effective modulus as well as the self-adaptive, superelastic, shape-memory properties discussed herein.

### Low-Cycle and Functional Fatigue in Shape Memory Alloys

As SMAs experience forward-reverse transformation cycles multiple times, their shape memory and superelastic properties undergo changes caused by defects that are generated and modified by the transformation. These changes may include an increase or decrease in transformation temperatures, reduction of stress, reduction of thermal hysteresis, accumulation of irrecoverable strain, and changes in the hardening rate of the stress-strain diagram. Traditionally, cyclic functional fatigue may be undesirable since it alters the properties of the alloy, causing it to deviate from its desired engineering response. However, functional fatigue can generally be stabilized after a number of transformation cycles, such that further cycles no longer affect properties appreciably, and in many applications. SMA components are subjected to such a stabilization process known as training to improve the predictability of the alloy response.

These transformation-induced changes are caused either by the generation of defects or retained martensite during transformation. As the interface between martensite and austenite form and move, defects are generated at the boundary because the lattice mismatch between the two phases, causing dislocations to form to relieve the accumulated internal stress. However, the precise effect of cyclic functional fatigue depends on the method by which the transformation occurs. When a SMA transform from changes in temperature under stress-free conditions, the transformation temperatures generally decrease. As transformation takes place, dislocations are generated at the interface between martensite and austenite. However, since no stress is applied, martensite is formed in the self-accommodated state and the stress field from the generated defects is randomly oriented. This means that no specific martensite variant is favored by the defects and as a result, the defects hinder the phase transformation by acting as obstacles to the movement of the phase front in subsequent transformation cycles. Austenite is stabilized over the martensite and transformation temperatures are decreased. On the other hand, if thermal-induced transformation occurs under applied stress, or if transformation is stress-induced, transformation cycles will increase the transformation temperatures of the alloy. Dislocations formed during these cycles are oriented and favors specific martensite variants, and thus the energy required to active the transformation of these variants are reduced, and martensite becomes stabilized compared to austenite. In structural implant applications, SMAs are used for their superelasticity. The focal point of present study is therefore the cyclic response of stress-induced phase transformation. Stress-induced phase transformation cycles cause three main changes in the stress-strain response of the alloy: 1) the transformation stress will gradually decrease with the number of cycles due to the increase in transformation temperature; 2) transformation stress hysteresis will be reduced, and 3) irrecoverable strain will be accumulated.

The aforementioned changes in the superelastic properties due to low-cycle functional fatigue have largely been explained by the generation of dislocations at the phase boundaries, similar to the explanation used for thermal-induced transformation of SMAs. However, retained martensite may play a role in the changes in superelastic behavior. When the material is loaded again, it no longer requires nucleation of martensite since martensite already exists in the matrix. Instead, the retained martensite simply grows. Since nucleation is reduced or eliminated, the energy barrier for the austenite→martensite transformation is significantly reduced, thus the stressed required to trigger stress-induced transformation is also reduced. The martensite is retained by dislocations, but other mechanisms, such as point defects and precipitates, have also been suggested as possibilities.

### Experimental Results Examples

In the experiments discussed below, the effect of superelastic cycling was used as a tool to reduce the effective modulus of the alloy. In order to create an ultra-low elastic modulus, alloys were fabricated from elements of 99.99% purity by vacuum arc melting and cast into rectangular billets of 0.75″×0.75″×5″. The billets were then solution treated at 1000° C. for 1 hour and water quenched. Two Ti—Nb-based shape memory alloy compositions were evaluated, a binary Ti₇₄Nb₂₆ (at. %) and a ternary Ti₇₂Nb₂₂Zr₆ (at. %) alloy. Equal channel angular extrusion (ECAE) of the Ti—Nb billets was performed using a 250-ton MTS hydraulic press with a custom extrusion tool constructed from Inconel 718, a precipitation hardened nickel-chromium alloy. The tool is an L-shaped channel with a sharp 90° angle with a square cross-section of 0.75″. Because of the low strength and superb ductility of the Ti—Nb and Ti—Nb—Zr alloys in the solution-treated state, the extrusion was easily carried out at room temperature. An extrusion rate to 0.01 in s⁻¹ allowed the extrusions to proceed without incident.

The binary Ti₇₄Nb₂₆ alloy was processed using two different ECAE routes. The first route (ECAE 1A) comprised one extrusion pass at room temperature, and the second route (ECAE 4Bc) comprised four extrusion passes with clockwise 90° rotation of the billet about the extrusion axis after each test at room temperature. The ternary Ti₇₂Nb₂₂Zr₆ alloy was extruded only via the first route at room temperature.

Shape memory and superelastic behavior did not appear in specimens extruded at room temperature without further heat treatments. A short intermediate temperature annealing process was then performed to allow some microstructure recovery. A plurality of heat treatments with temperatures between 400° C. to 800° C. and durations between 5 minutes to 1 hour were carried out on the as-extruded specimens, and all specimens were water-quenched. Table 1 summarizes all the post-extrusion annealing heat treatments studied that showed promising shape memory properties and reflects that an additional precipitation heat treatment at 300° C. for 1 hour was performed on the annealed specimens A610 and C610, the alloy notations are correlated below to the mechanical process and heat treatment received by each sample.

TABLE 1

| Mechanical Process | Heat Treatment | Notation |
|---|---|---|
| ECAE 1A | 600° C. 10 minutes | A610 |
| | 600° C. 1 hour | A61H |
| ECAE 4Bc | 600° C. 10 minutes | C610 |
| | 600° C. 5 minutes | C65 |
| | 500° C. 10 minutes | C510 |

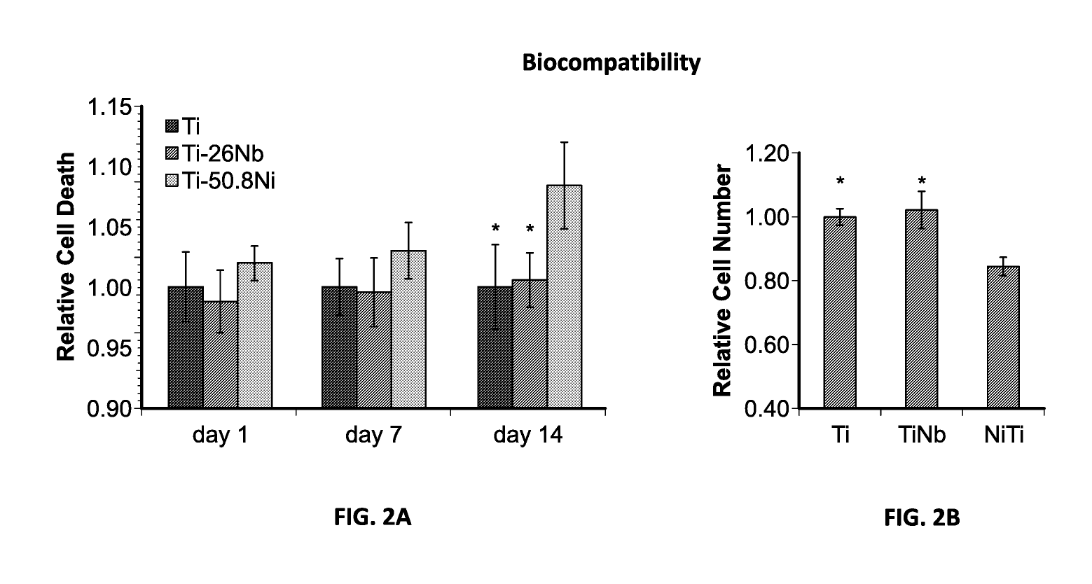

In addition to the mechanical properties and behavior desired from these alloys, properties such as biocompatibility and corrosion-resistance are preferred and may be necessary, as such, cytoxicity and corrosion results are discussed herein. FIGS. 2A and 2B show the result of the cytotoxicity experiments on pure titanium, Ti₇₄Nb₂₆, and Ni_{50.8}Ti₄₉. The results indicate that the Ni_{50.8}Ti₄₉ SMAs were somewhat more toxic than the Ti₇₄Nb₂₆ and pure Ti samples, whereas Ti₇₄Nb₂₆ and Ti were similar in their cytocompatibility. The cytotoxicity of the corrosion products varies widely for cells of different organisms. For example, exposure of human blood lymphocytes to bulk Ni—Ti surface treated by autoclaving in steam or water evoked little adverse cellular response, while rat blood lymphocytes exposed to similarly processed Ni—Ti actually showed an improved stimulation in cell proliferation. On the other hand, when exposed to porous Ni—Ti, which has higher surface area, the proliferation of the rat lymphocyte was almost completely suppressed, similar to the effect of pure nickel, while the reduction of human lymphocytes is only about 30%. The cells (NIH/3T3) used in the present study is a mouse fibroblast line, so it is possible that for human cells, the cytotoxicity response may be different. One certainty among the sometimes contradictory findings on the cytotoxicity of Ni—Ti in published literature is that higher nickel release and higher concentration of nickel atoms at the surface of the alloy are correlated with reduced cell viability. Therefore, it is important to concentrate on the specific mechanism of metal release on cytotoxicity; this is the reason that we chose to conduct an indirect contact study. In this case, the results are expected to depend only on the identity and amount of metallic corrosion products and minimize the direct effect of surface features on cell viability since cells do not come in direct contact with the specimen. Since both the Ni—Ti and Ti—Nb specimens were polished to the exact same final finish (0.05 μm), the surface roughness, the surface area of the two specimens are expected to be similar.

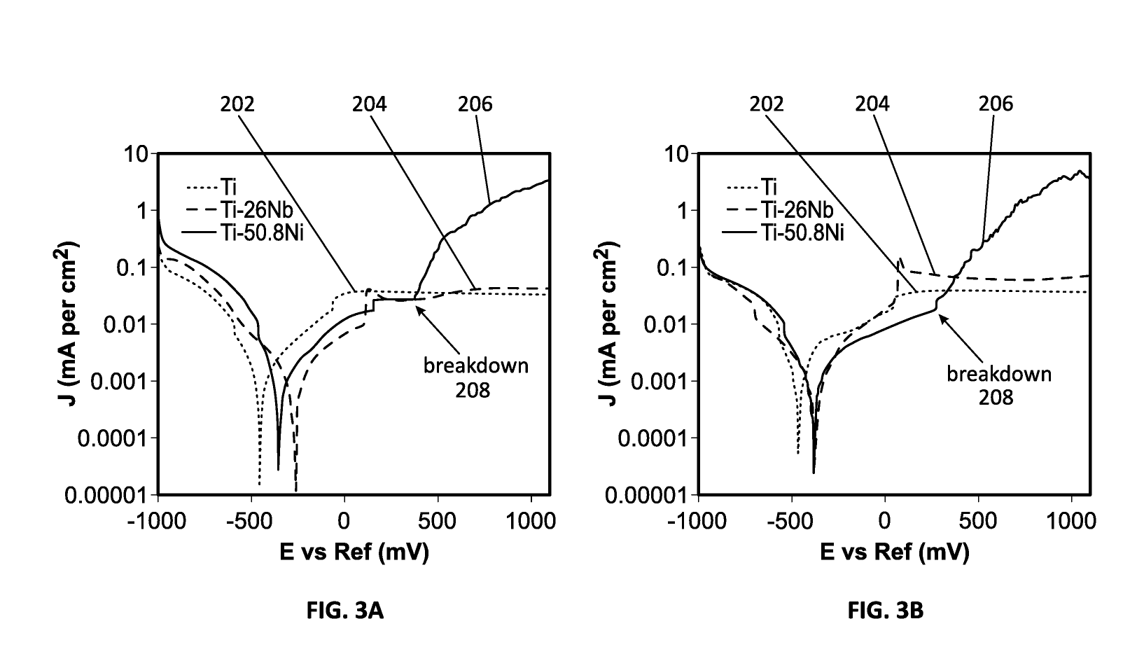

FIGS. 3A and 3B are potentiodynamic polarization graphs depicting the reaction of pure Ti 202, Ti-26Nb 204, and Ti-50.8Ni 206, in a saline (3A) and modified Hank's solution (3B) environment. Potentiodynamic tests were performed at 37° C. on Ni_{50.8}Ti_{49.2}, Ti₇₄Nb₂₆, and commercial pure Ti samples submerged in 1M NaCl or in Hank's Balanced Salt Solution (HBSS; 8 g NaCl, 0.4 g KCl, 0.14 g CaCl₂, 0.06 g MgSO₄, 0.06 g NaH₂PO₄, 0.35 g NaHCO₃, 1 g glucose, 0.6 g KH₂PO₄, 0.1 g MgCl₂ per liter de-ionized water). Corrosion resistance is a preferable property and characteristic of implantable alloys. In some cases, corrosion resistance similar to pure titanium (Ti) is desired. In order to test the corrosion resistance of each alloy, the alloys were submerged in a solution, a current was applied as indicated along the Y-axis, and a potential was measured as indicated along the X-axis. The corrosion resistance in FIG. 3A represents the effects of a corrosive environment that uses saline. FIG. 3A shows that the polarization curve of Ti-26Nb 204 immersed in 1M NaCl demonstrated a passive response similar to that of Ti over the potential range examined. In contrast, Ni_{50.8}Ti_{49.2} 206 specimens submerged in 1M NaCl showed a breakdown potential at 400 mV relative to the reference electrode. Turning to FIG. 3B, Hank's modified solution, used in this test, is a balanced salt solution (HBSS) used for corrosion resistance testing because it may be applied to an instrument, implant, or to cell tissue such as allographs during the manufacture or transport of that product. As such, testing these alloys for corrosive behavior in Hank's solution may be prudent to ensure the alloys maintain integrity during processing and transport. In FIG. 3B, Hank's modified solution is used which means that the solution does not have Ca++ or Mg++. The bare surfaces of the Ti—Nb SMA did not suffer from breakdown up to 2000 mV, which may suggest that the inherent corrosion resistance of Ti—Nb is equal to or better than that of Ni—Ti. It also means that the corrosion resistance of Ti—Nb is less sensitive to surface conditions than that of Ni—Ti, and that Ti—Nb does not require the same level of stringent surface processing as needed for Ni—Ti to maintain excellent corrosion resistance. It is noted that, while the Ti—Nb—Zr alloy was not tested during the corrosion testing depicted in FIGS. 3A and 3B, it is appreciated that zirconium may be used as an alloying agent because of its corrosion resistance. As such, one skilled in the art would appreciate that adding Zr, which may be used for its corrosion resistance, to a Ti—Nb alloy that has demonstrated corrosion resistance would not negatively impact the corrosion resistance of that alloy.

Superelastic properties were evaluated from a plurality of loading-unloading cycles during experiments. Tension specimens were used to characterize the superelastic response of the materials. Specimens were loaded at a constant temperature to a certain strain level, unloaded, and then loaded again to a higher level of strain. This process continues until significant irrecoverable strain is detected after unloading, which generally occurred at 2.5% to 3% applied strain levels.

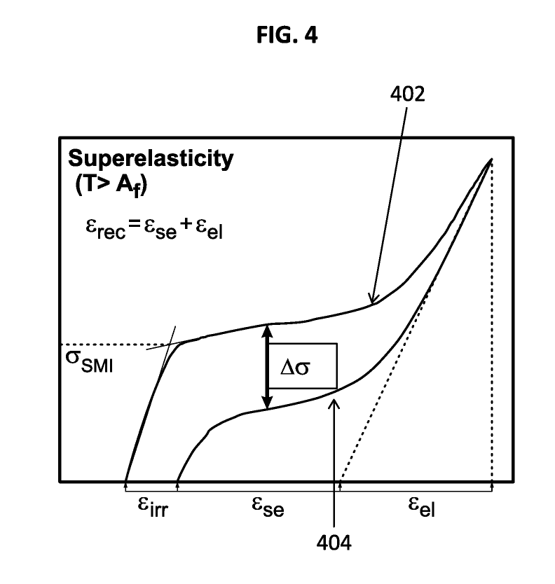

FIG. 4 is a graph of superelastic properties exhibited by Ti—Nb. In FIG. 4, σ_{SIM} denotes the critical stress for stress-induced martensitic transformation; εᵢᵣᵣ, εₛₑ, and εₑₗ represent irrecoverable strain, superelastic shape strain, and elastic recoverable strain, respectively. Total strain recovery (ε_{rec}) in superelasticity is the sum of εₛₑ and εₑₗ.

Important superelastic properties, shown in FIG. 4 are primarily irrecoverable strain (εᵢᵣᵣ), recoverable strain (ε_{rec}), and critical stress for stress-induced transformation (σ_{SIM}). Recoverable strain includes elastic recovery and recoverable shape change from the stress-induced martensitic transformation and possibly also martensite detwinning. With increasing applied strain, both ε_{rec} and εᵢᵣᵣ tend to increase. ε_{rec} reaches a maximum at some strain level while εᵢᵣᵣ increases monotonically with applied strain. The stress hysteresis Δσ is the stress difference between the loading 402 of the stress-strain curve and the unloading 404 part of the stress-strain curve.

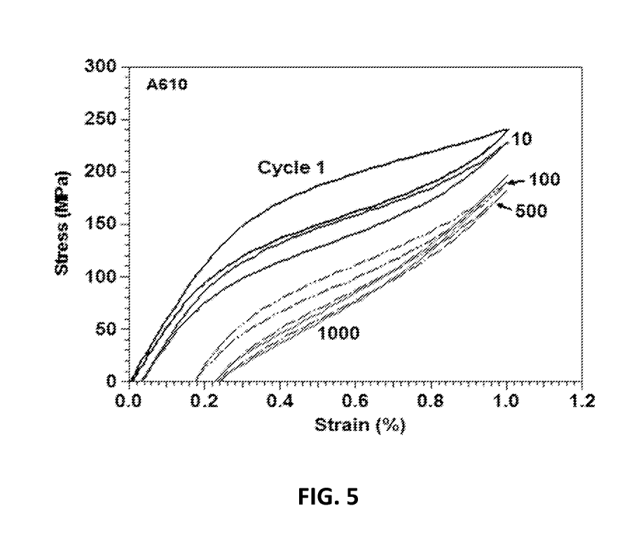

FIG. 5 is a graph of multiple stress strain curves where the Ti—Nb alloy was cycled for varying numbers of cycles. During constant-temperature superelastic cycling, a number of superelastic properties changes, as demonstrated in FIG. 4. With increasing number of cycles, stress hysteresis (Δσ) decreased, and irrecoverable strain (εᵢᵣᵣ) increased in the initial stages of cycling.

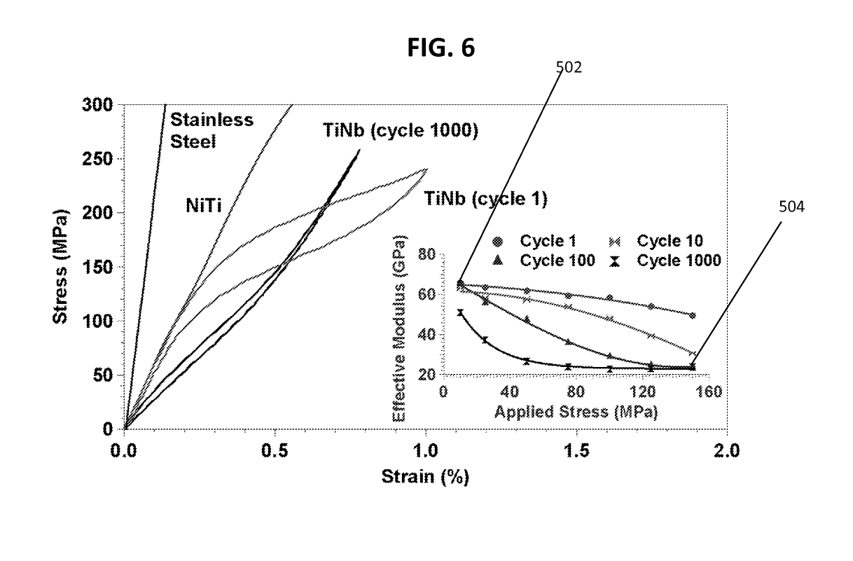

FIG. 6 is a graph of the stress-strain behavior of various alloys and the inset graph shows the effect of the applied stress level on the effective modulus of 1, 10, 100, and 1000 cycles. Also shown in FIG. 6 is the stress-strain behavior of the Ti—Nb alloy before and after cycling in comparison to other metallic materials. Prior to cycling; the elastic modulus of Ti—Nb is about 65 GPa indicated by 502, similar to that of the Ni—Ti in the austenite state, and about ⅓ of value of stainless steel. On the other hand, after 1000 cycles, the effective modulus, also referred to as the secant modulus, is further reduced 504 due to the early introduction of stress-induced phase transformation.

### Cyclic Superelastic Response and Low-Cycle Fatigue

Several specimens were subjected to a constant-strain, constant-temperature cyclic superelastic experiments. Here, the specimens are loaded to 1% or 1.5% strain 1000 or 1500 times at room temperature to evaluate the low-cycle functional fatigue properties of the alloys. The transformation stress (σ_{SIM}), stress hysteresis (Δσ), and irrecoverable strain (εᵢᵣᵣ) as a function of number of superelastic cycles are summarized.

These experiments ultimately led to the creation of a method to reduce the effective elastic modulus of the disclosed alloys. The method developed combines elastic deformation with another reversible deformation mode to create a reduced effective modulus without affecting the true elastic modulus or other mechanical properties of the alloy. By combining elastic deformation and reversible stress-induced phase transformation in both the Ti—Nb and Ni—Ti shape memory alloys, an apparent effective modulus of below 30 GPa was achieved as shown in FIG. 6. Furthermore, this method enables the alloy to automatically adjust its apparent effective modulus to the properties of the surrounding bone: when the surrounding bone weakens, the apparent effective modulus of the material will be reduced in response, and vice versa.

Conventionally, a relatively high stress level is needed to trigger the stress-induced transformation. This stress level is too high for the natural load state of the body to trigger the transformation. Through superelastic cycles, it is possible to reduce the transformation stresses to very low levels. The Ti—Nb A610 alloy was cycled 1000 times at room temperature to 1% strain. As discussed above, FIG. 5 shows the resulting stress-strain curves. FIG. 6 is a comparison of the stress-strain response of stainless steel, Ni—Ti shape memory alloy, and the Ti₇₄Nb₂₆ A610 shape memory alloy before and after 1000 superelastic cycles. The inset shows the effective modulus (secant modulus) of the Ti—Nb SMA after various numbers of superelastic cycles as a function of stress level.

The reduced effective modulus is enabled by the stress-induced phase transformation which takes place at a very low σ_{SIM}. The effect of cycling reduced the σ_{SIM} level at cycle 1000 to about 20 MPa, and at this point, the stress-strain curve deviates from linearity and the slope is gradually reduced. At about 75 MPa, the stress-strain curve of the cycle 1000 specimen reaches an inflection point and the slope begin to increase once again. This means that the effective modulus is not only a function of the number of superelastic cycles, but also a function of the stress level. The inset of FIG. 6 shows these relationships: as applied stress and number of superelastic cycles increase, the effective modulus drops from the original value of nearly 65 GPa to a minimum of about 22 GPa at about 75 MPa in cycle 1000. This method of modulus reduction may be referred to as “cyclic softening”.

Stress shielding, as discussed above, occurs because the implant material carries a larger portion of the load than the surrounding bone, due to the higher elastic modulus of the implant. For implant constructed of conventional metals and alloys, the effect of stress shielding will naturally intensity as bone resorption and loss leads to further transfer of load to the implant, and accelerates the bone loss process. However, in the cyclically-softened Ti—Nb SMA, if bone loss occurs that leads to the transfer of more loads to the implant, the effective elastic modulus of the implant will be reduced and return the load back to the bone and preventing further degradation. The stress-dependence of the effective modulus allows the implant to adjust its properties based on its operating environment: if a higher than desired level of load is carried by the implant, it will automatically reduce its effective modulus to transfer load back to the surrounding.

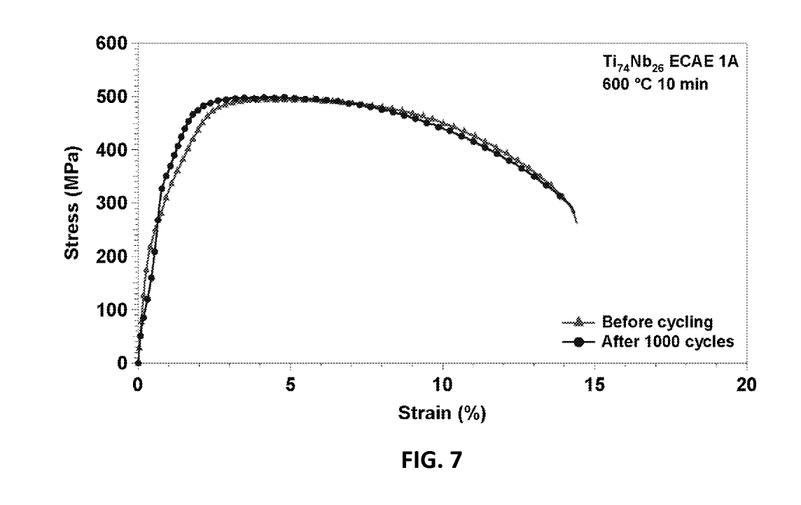

Finally, the stress-induced transformation approach, which may also be referred to as training, allows the effective modulus of the material to be reduced without affecting other mechanical properties, as shown in FIG. 7. FIG. 7 is a stress-strain diagram of the A610 alloy before and after 1000 superelastic cycles. Although cycling reduces the effective modulus, it does not affect the mechanical properties of the material. However, stress of bones in the human body from everyday activities is very difficult to measure as different bones may experience stress differently and it may vary from person to person depending on gate, age, activity level, weight, fitness level, nutrition, and other factors such as genetics. In metallic materials, the elastic modulus of a material and its ultimate strength are directly correlated, that is, stiffer materials are generally stronger. Thus the act of reducing the elastic modulus is often accompanied by a reduction in ultimate strength, and thus fatigue resistance—both parameters crucial to implants designed for long operating lifetimes. Similarly, the low elastic modulus of porous materials comes at the cost of strength as well. However, the stress-induced transformation approach does not actually change the inherent elastic constant of the material, but rather introduces a second reversible deformation mechanism to reduce the effective modulus. This means that the inherent material properties, such as strength, are not adversely affected. The effective modulus may be reduced without compromising other properties.

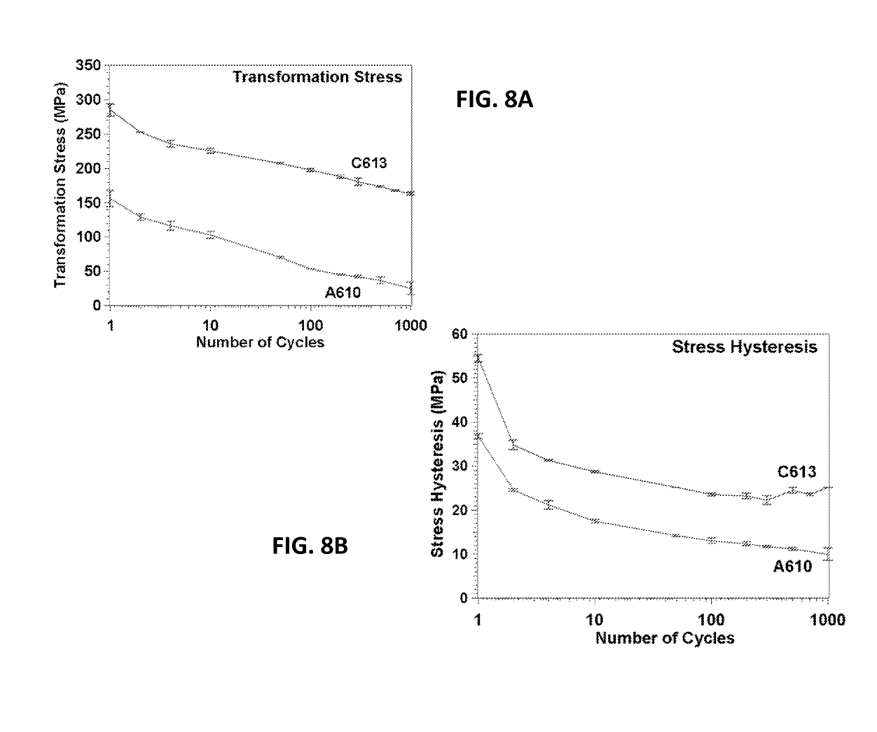

However, the effectiveness of softening may be affected by the initial condition of the alloy. FIGS. 8A-8B show the relationship between σ_{SIM} and number of cycles in the A610 and C613 specimens. While both experiences reduction in σ_{SIM} of C613 remains above 150 MPa after 1000 cycles. Since the reduction in effective modulus depends on reducing σ_{SIM} to very low levels, the modulus of C613 does not change until the applied stress level exceeds 150 MPa. It should also be noted that the transformation stress values achieved through cycling are lower than what is normally possible through changing the superelastic temperature relative to the transformation temperature of the material. In the austenite state, the transformation stress is directly proportional to the temperature at which the material is deformed. Thus, if the experiment is carried out at temperatures closer to the austenite finish (A_{f}) of the material, the transformation stress is naturally lowered.

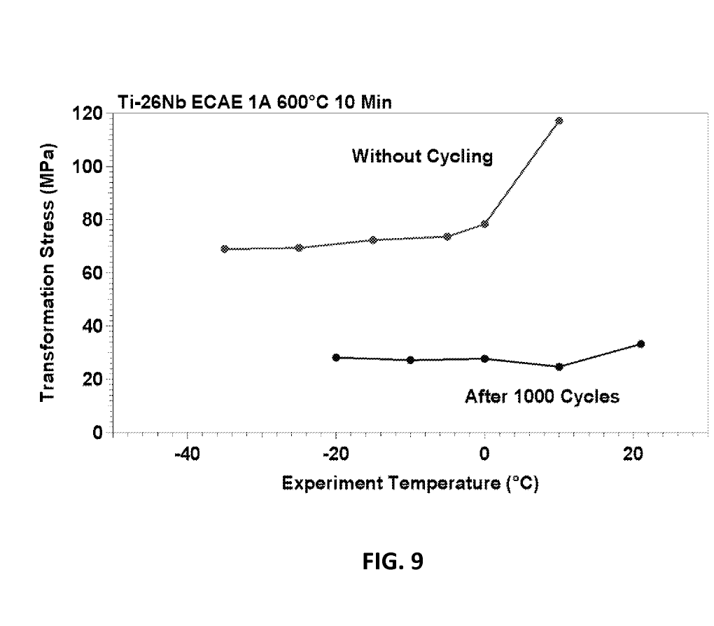

This lowering is shown in FIG. 9 which shows the dependence of transformation stress on the testing temperature in the A610 specimen before and after 1000 superelastic cycles, specifically, that the transformation stress is lowered after 1000 cycles and does not spike around 12° C. like the sample tested without cycling. In FIG. 8A, σ_{SIM} of C613 is higher than that of A610, implying that the ω precipitates, smaller grain size and higher dislocation density of C613 stabilized the austenite relative to martensite and effectively lowered the transformation temperatures of the alloy. The lower the transformation temperature, the higher the σ_{SIM} would be at room temperature. Both precipitates and dislocations hinder the movement of martensite interface during transformation, thus increasing the total energy demand of the transformation and increasing σ_{SIM}. Simultaneously, σ_{SIM} is also affected by the change in chemistry from precipitation. The w precipitates are rich in titanium, so their appearance increases the niobium concentration in the matrix. A higher niobium concentration lowers transformation temperatures, which further raises σ_{SIM} in precipitated samples. Over the course of 1000 superelastic cycles to 1% strain, the σ_{SIM} of both specimens decreased by about 130 MPa. The initial Δσ is also higher in C613 as shown in FIG. 8B, and Δσ of both specimens decreases with increasing number of cycles. Δσ is a measure of energy dissipation and it is caused by interfacial friction and creation and/or rearrangement of defects during the martensitic transformation. The effect of defect generation and rearrangement is usually high in the first cycle, and becomes less significant with each additional cycle. Therefore, the change in Δσ is most pronounced in the first few cycles and then Δσ tends to saturate.

However, σ_{SIM} values below 100 MPa are very difficult to achieve by changing the experiment temperature alone. By comparison, for a specimen cycled 1000 times to 1% strain, the minimum achievable transformation stress varies only weakly with testing temperature and remains much lower than the specimen without cycle at all temperatures. This means that cyclic softening is required to achieve the ultra-low elastic modulus of the material. One practical problem with the cyclic softening technique is the amount of cycles required to achieve the desired level of σ_{SIM}. It is neither economical nor practical to apply 1000 cycles individually to each device, and a simpler method is needed.

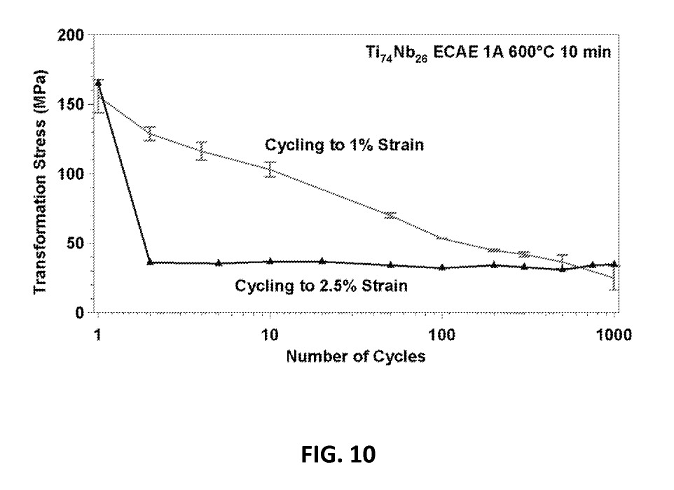

FIG. 10 is a graph that illustrates how a high number of superelastic cycles at a low strain can be replaced by a lower number of cycles at a higher strain. This may be an economical way to produce the same ultra-low effective modulus with a fraction of the cycles in the training/cyclical softening process. FIG. 10 shows how 1000 superelastic cycles at 1% strain were replaced by 1 cycle at a high strain level to produce an equivalent result. When the specimen (A610) was cycled once to 2.5% strain, the transformation stress level was reduced from 150 MPa to about 30 MPa immediately, thus greatly simplifying the cyclic softening process.

Through these experiments, a stress-induced transformation technique was developed via cyclic softening to create alloys with ultra-low effective elastic modulus below 30 GPa, about half the value of the lowest reported stiffness value of 55 GPa in biomedical alloys, without negatively affecting other mechanical properties of the material. The shape of the stress-strain curve of the material further allows the material to self-adjust to the condition of the surrounding both in order to more effectively combat stress shielding. This technique may be able to be applied to all SMAs that demonstrate superelasticity.

### Methods of Manufacture

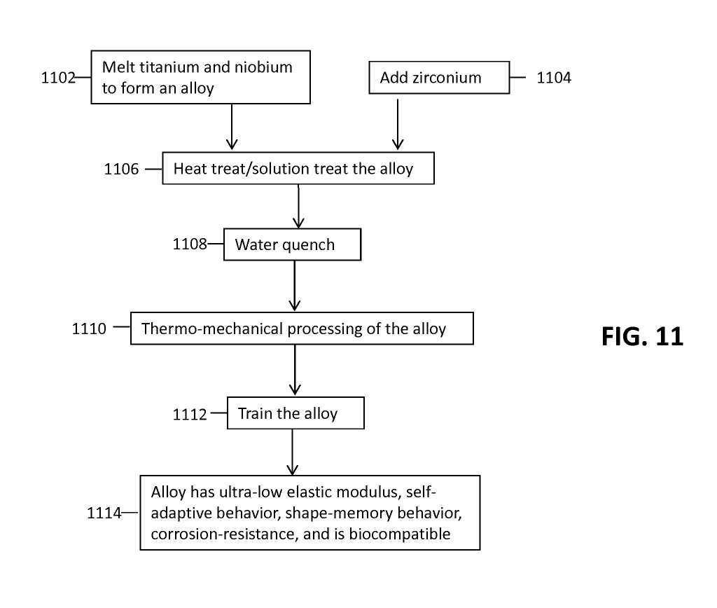

FIG. 11 is a method of manufacturing an ultra-low effective elastic modulus with shape memory and superelastic properties. At block 1102, titanium (Ti) is melted with niobium (Nb) in, for example, a vacuum arc furnace. In this embodiment, the atomic percent of Nb is between 23%-26% and the balance is Ti. The alloy is then heat treated or solution treated at block 1106 which may be followed by a water quench at block 1108. In one example, the heat treatment comprises holding the alloy between 850° C.-1100° C. for a minimum of 30 minutes. In other examples, the heat treatment may comprise holding the alloy between 400° C.-800° C. or between 450° C. to 600° C. treatments 5 minutes to 60 minutes, followed by a water quench at block 1108.

Alternatively, the heat treatment at block 1106 may last from 30 minutes-72 hours. After heat treatment at block 1106, which may comprise one or more heat treatment steps and a water quench at block 1108, the alloy may be thermo-mechanically processed by hot, cold, or warm deformation including extrusion, rolling, swaging, equal channel angular extrusion, and wire drawing. The alloy may demonstrate a self-adaptive, low-effective modulus response subsequent to heat treatment but prior to thermo-mechanical processing. However, the fatigue life and strength levels of those alloys may be lower than those which have been thermo-mechanically processed. Subsequent to heat treatment at block 1106 and a water quench at 1108, the alloy is thermo-mechanically processed at block 1110. The thermo-mechanical processing used at block 1110 may depend on the end application of the alloy and may comprise one or more process conducted at one or more temperatures or temperature ranges. In an embodiment, subsequent to a cold-working process, the alloy undergoes a second heat treatment process, wherein the second heat treatment process comprises holding the alloy between 200° C. and 700° C. for at least 30 minutes and up to 100 hours While implants and orthopedics in general are discussed herein, it is noted that the alloys disclosed can be used for any application where corrosion resistance, super elasticity, self-adaptiveness, ultra-low modulus of elasticity, and shape-memory are desirable properties. This includes but is not limited to aerospace, automotive, and automation where components of a system may be subjected to corrosive environments, cyclic loading, high strains, or a combination of factors. In one example, the thermo-mechanical process comprises more than 10% of Von mises equivalent strain, and in an alternate example, the thermo-mechanical process comprises more than 50% of Von mises equivalent strain.

Turning back to FIG. 11, after the thermo-mechanical processing at block 1110, which may include post-processing heat treatment and a subsequent water quench, the alloy is trained at block 1112. Training is the process wherein a load is applied cyclically to the alloy in order to create the desired properties. This training at block 1112 may be performed at room temperature or at an elevated temperature of up to 150° C. The strain level used during training may be 1%-5% strain. It is appreciated that, as shown and discussed above in FIG. 10, in some embodiments up to 1000 superelastic cycles may be performed at 1% strain to produce an elastic modulus of about 30 GPa and in an alternate embodiment one cycle at a higher strain level such as 2.5% can also produce an equivalent modulus. In one example, the training comprises cyclic loading on the alloy to strains larger than 0.5% strain at a temperature where the alloy exhibits superelastic behavior, for example, at a temperature below 150° C. In an alternate example, the training comprises cyclic loading on the alloy to 1-3% strain at 10° C. (50° F.)-100° C. (212° F.), preferably between 20° C. (68° F.)-50° C. (122° F.). The training process at block 1112 may be dependent on the composition of the alloy as well as the desired end use. The resultant alloy at block 1114 will have corrosion resistance equivalent to pure Ti, an effective modulus of elasticity <30 GPa, and demonstrate self-adaptive, superelastic properties. It is understood as discussed above with respect to FIG. 10, that the greater the strain, the % strain, of the training, the fewer training cycles may be needed.

In an alternate embodiment, a ternary alloy, for example, zirconium, may be added at block 1104. In this example, the atomic % of Ti is between 66%-76%, the atomic % of Nb is between 20%-26% and the atomic % of Zr is between 4%-8%. In another example, the total effective content (atomic %) of Nb+Zr is between 24%-26%. In this example, for every 1% of Nb there is, there would be 0.67*Zr at % and the total of (1*Nb at %)+(0.67+Zr at %)=24%-26% total, where the balance is Ti. In either example where Zr is added at block 1104, the method in FIG. 11 proceeds as described above from blocks 1106-1114. The heat treating at block 1106 and the thermo-mechanical processing at block 1110 may proceed at above or may have varied parameters and involved more than one station at one or both block 1106 and block 1110. At block 1114, the alloy will have an effective modulus of <30 GPa and exhibits self-adaptive, shape-memory, and superelastic behavior.

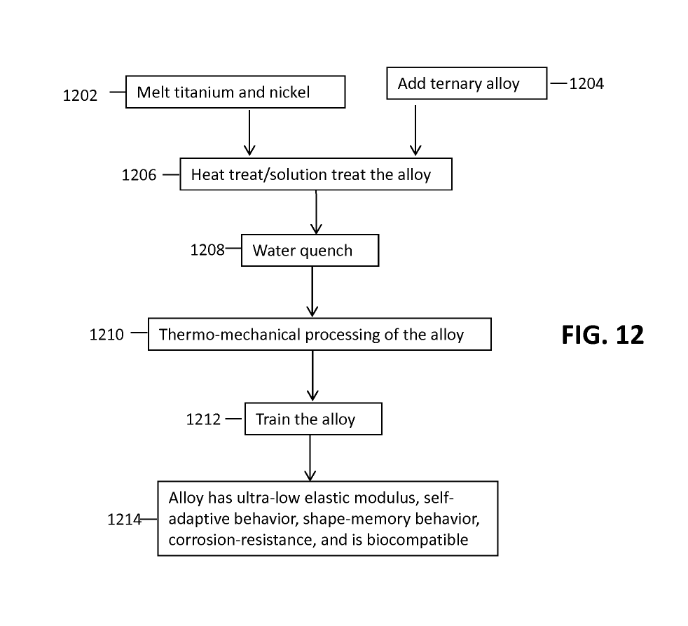

FIG. 12 is an alternate embodiment of a method of manufacturing a self-adaptive shape-memory alloy with an ultra-low effective modulus of elasticity. At block 1202, Ti and Ni are melted under vacuum. In some embodiments, a ternary alloy is added at block 1204. In some embodiments, two components such as Ti, Ni, or the ternary alloy may be used as pre-made ingots and melted under vacuum, i.e., each type of material (metal) may be melted separately to form the alloy or two or more types of material (metals) already in ingot or other usable form may be melted with an at least an additional metal. In the absence of a ternary alloy added at block 1204, the atomic % Ni is between 49.5%-55 and the balance is Ti. The alloy formed at block 1202 is then heat treated or solution treated at block 1206 which may be followed by a water quench at block 1208. In one example, the heat treatment comprises holding the alloy between 700° C.-1100° C. for a minimum of 30 minutes. In other examples, the heat treatment may comprise holding the alloy between 400° C.-700° C. or between 250° C. to 600° C. treatments 5 minutes to 60 minutes, followed by a water quench at block 1208. Alternatively, the heat treatment at block 1206 may last from 30 minutes-100 hours. After heat treatment at block 1206, the alloy may be thermo-mechanically processed by hot, cold, or warm deformation including extrusion, rolling, swaging, equal channel angular extrusion, and wire drawing. In one embodiment, subsequent to a cold-working process, the alloy undergoes a second heat treatment process, wherein the second heat treatment process comprises holding the alloy at 200° C. to 500° C. for at least 30 minutes and up to 100 hours. It is understood that a treatment at a higher temperature may require to a shorter heat treatment time. While the experiments and findings herein were performed in a research environment, it is appreciated that processing steps that may reduce the overall processing time required to produce the desired alloy may be desired by industry for cost, safety, and efficiency purposes.

The alloy may demonstrate a self-adapative, low-effective modulus response subsequent to heat treatment but prior to thermo-mechanical processing. However, the fatigue life and strength levels of those alloys may be lower than those which have been thermo-mechanically processed. Therefore, the alloy is thermo-mechanically processed at block 1210. The thermo-mechanical processing used at block 1210 may depend on the end application of the alloy and may comprise one or more process conducted at one or more temperatures or temperature ranges. In one example, the thermo-mechanical process comprises more than 10% of the Von mises equivalent strain for the alloy system, and in an alternate example, the thermo-mechanical process comprises more than 50% of the Von mises equivalent strain.

Turning back to FIG. 12, after the thermo-mechanical processing at block 1210, the alloy is trained at block 1212. Training is the process wherein a load is applied cyclically to the alloy in order to create the desired properties. This training at block 1212 may be performed at room temperature or at an elevated temperature of up to 150° C. s. The strain level used during training may be 1%-5% strain. In one example, the training comprises cyclic loading on the alloy to strains larger than 0.5% strain at a temperature where the alloy exhibits superelastic behavior. The training process at block 1212 may be dependent on the composition of the alloy as well as the desired end use. The resultant alloy at block 1214 will have corrosion resistance equivalent to pure Ti, an effective modulus of elasticity <40 GPa, and demonstrate self-adaptive, shape-memory behavior and superelastic properties. As discussed above with respect to FIG. 10, the greater the strain, that is, the greater the % strain, used for the training, the fewer cycles may be needed.

Embodiments of the spinal implant can have surface modifications, in the form of protruding members, on the top and bottom surfaces configured to enhance stability and anchoring of the spinal implant between adjacent vertebrae. In at least one embodiment, the top and bottom surfaces of the spinal implant are in the form of a keel. The cross-sectional profile of the keel may have different shapes. For instance, the cross-sectional profile of the keel may have the shape of a wedge, a truncated wedge, a triangle, a truncated triangle, a rectangle, or a square. In alternate embodiments, of the present disclosure, the protruding members can be a rough surface, and/or a surface comprising ridges, spikes, raked or straight teeth, protrusions or any combination thereof. The protruding members can be made of the same shape memory alloy as the spinal implant, a different shape memory alloy, a biocompatible metal, or any other suitable material. It will be understood by those skilled in the art that the above described shape memory alloys can be fabricated with any suitable surface modifications and the surface modifications described herein are not intended to limit the scope of this disclosure.

In alternate embodiments, the external surfaces of the spinal implant are solid and smooth. In alternative embodiments of the present disclosure, the external surface of the spinal implant can be roughened or corrugated, or have one or more recesses or apertures running therethrough.

Embodiments of the spinal implant can be constructed in part from any biocompatible material, including synthetic or natural autograft, allograft, or xenograft tissues, and can be resorbable or non-resorbable in nature. Tissue materials can include, for example, hard tissues, connective tissues, demineralized bone matrix, and combinations thereof. Resorbable materials such as, for example, polylactide, polyglycolide, polyorthoester, polyphosphazene, tyrosine-derived polycarbonate, bioactive glass, calcium phosphate, hydroxyapatite, and combinations thereof may also be used. Non-resorbable materials such as, for example, non-reinforced polymers, carbon-reinforced polymer composites, PEEK and PEEK composites, ceramics, and combinations thereof may also be used. It will be understood by those skilled in the art that the above described shape memory alloys can be constructed in part with any suitable biocompatible materials and the biocompatible materials described herein are not intended to limit the scope of this disclosure.

For some embodiments of the spinal implant it may be advantageous, in some circumstances, to provide the spinal implant with a suitable osteogenic material and/or therapeutic composition. Suitable osteogenic materials can include, for example, autograft, allograft, xenograft, demineralized bone, synthetic and natural bonegraft substitutes, such as bioceramics, polymers and osteoinducive factors. These materials may be provided into or onto the spinal implant where surfaces are roughened or corrugated, or in recesses or apertures running therethrough. A separate carrier such as, for example, collagen-based carriers, bioceramic materials, calcium phosphate, hydroxyapatite, or any combination thereof, can be used to hold the osteogenic materials in their desired location. The carriers can also be partially comprised of therapeutic or infection resistant agents. The carriers can also be partially comprised of an effective amount bone morphogenic protein, transforming growth factor 1, insulin-like growth factor 1, platelet-derived growth factor, fibroblast growth factor, LIM mineralization protein (LMP), and any combinations thereof. It will be understood by those skilled in the art that the above described shape memory alloys can be fabricated with any suitable osteogenic material and/or therapeutic composition and the osteogenic materials and therapeutic compositions described herein are not intended to limit the scope of this disclosure.

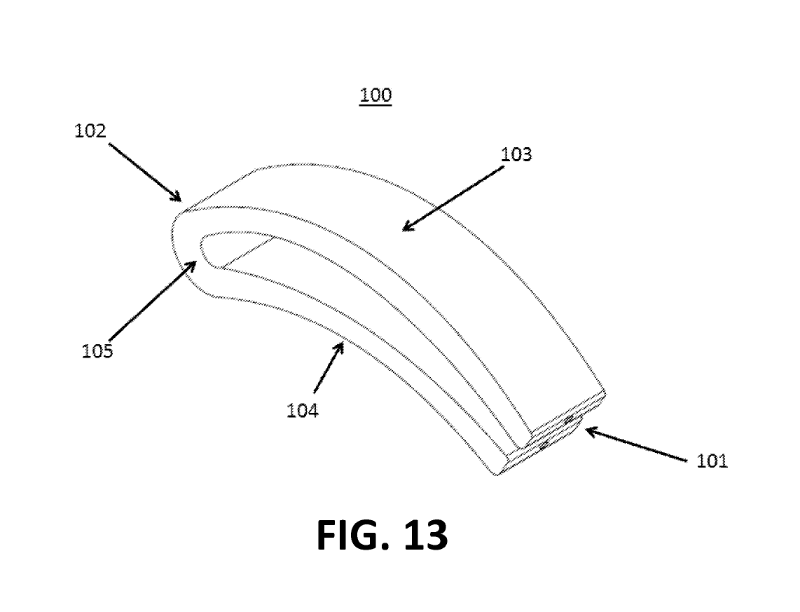

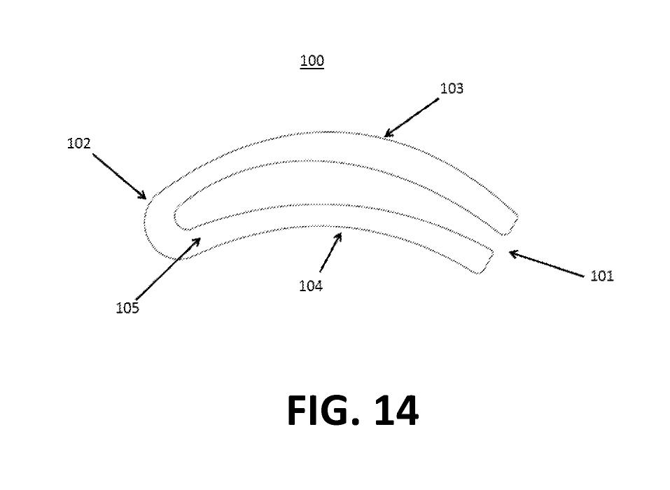

FIGS. 13 and 14 illustrate an exemplary embodiment of a shape memory alloy spinal implant 100 in a shrunken state for insertion into a cavity of a patient between adjacent vertebrae. As shown, in the shrunken state the spinal implant 100 is substantially in the shape of a hairpin. The spinal implant 100 is defined by an open end 101 and closed end 102. The open end 101 is configured to couple to an insertion rod (not shown) which facilitates implantation into a patient. The spinal implant further comprises a convex surface 103 and a concave surface 104. The concave surface 104 is configured to face the spinal cord of the patient. Upon insertion in a patient, a bottom surface 105 and a top surface (not shown) opposite the bottom surface are configured to engage the adjacent lower and upper vertebrae respectively.

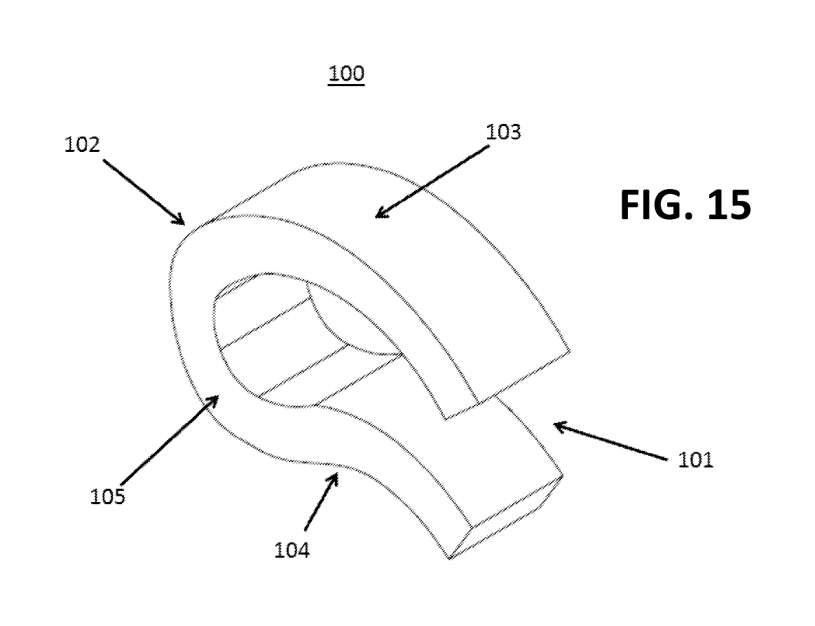

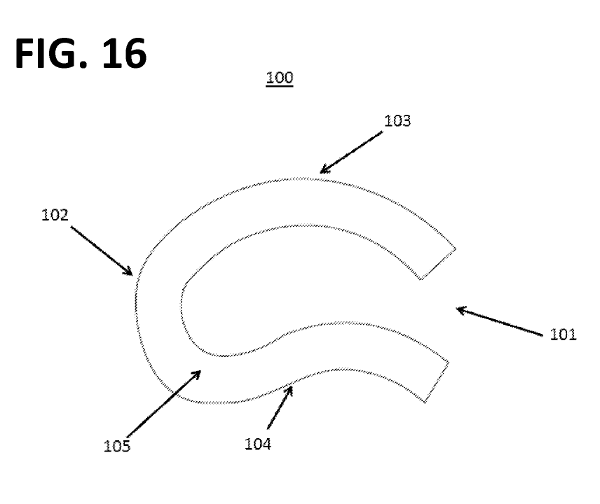

FIGS. 15 and 16 illustrate the exemplary shape memory alloy spinal implant 100 of FIG. 13 in an expanded state after insertion into a cavity of a patient between adjacent vertebrae. As shown, during transformation from the shrunken state to the expanded state, the spinal implant 100 transforms from a substantially hairpin shape to a substantially C-shaped disc implant 100. Here, the expanded spinal implant 100 is described as substantially C-shaped meaning that the spinal implant resembles a C-shape, but deviates from a true C-shape with respect to the concave surface 104. The expanded spinal implant 100 can be described as exhibiting an overall shorter length from an outer surface of the open end 101 to an outer surface of the closed end 102 compared to the shrunken state. The expanded spinal implant can also be described as exhibiting larger thicknesses along cross-sections parallel and perpendicular to the bottom surface 105. Furthermore, the general shape of the convex surface 103 and the concave surface 104 are retained while the degree of curvature is more pronounced. The concave surface can also be defined as also having a concave surface and convex surface wherein the concave surface is oriented toward the open end 101 and the convex surface is oriented toward the closed end 102.

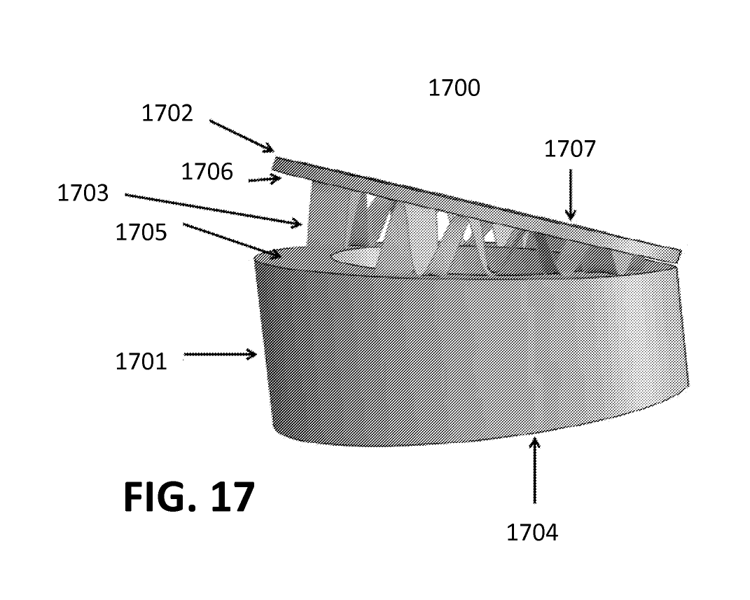

FIG. 17 illustrates an isometric view of another exemplary embodiment of a shape memory alloy spinal implant 1700. The spinal implant 1700 can be used in a patient requiring replacement of a compromised spinal exhibiting lordosis or other irregular curvatures of the spine. The spinal implant 1700 includes a substantially cylindrical main implant body 1701 having a bottom surface 1704, a top surface 1705 and a substantially circular aperture extending vertically therethrough. The spinal implant 1700 further includes a disc 1702 that is substantially the same diameter as the main implant body 1701. The disc 1702 includes bottom surface 1706 and a top surface 1707 and is acutely angled relative to the main implant body 1701. The main implant body 1701 and disc 1702 are connected by one or more support structures 1703 connected to the top surface 1705 of the main implant body 1701 and the bottom surface 1706 of the disc 1702. As shown in FIG. 17, the support structures 1703 are two ribbons beginning at the closest point between the main implant body 1701 and disc 1702, oscillate along the acute angle formed between the main implant body 1701 and disc 1702, and connect the main implant body 1701 and disc 1702 at points of local minima and local maxima of each oscillating ribbon. In alternate embodiments, the one or more support structures 1703 can be individual strips connected to the top surface 1705 of the main implant body 1701 and the bottom surface 1706 of the disc 1702. The angle between the main implant body 1701 and disc 1702 can be altered by changing the relative heights of the one or more structural supports 1703.

In FIG. 17, the main implant body 1701, the disc 1702 and the structural supports 1703 are made of the same any one the shape memory alloys disclosed above. In at least one embodiment, the main implant body 1701, the disc 1702 and the structural supports 1703 can be made of different shape memory alloys to take advantage of different transformation characteristics of each respective shape memory alloy. The differing transformation characteristics may be for example, but not limited to, different transformation temperatures, degrees of expansion, or direction of expansion relative to the spinal column. In at least one embodiment, only the structural supports 1703 are made of a shape memory alloy while the main implant body and disc are made of non-shape memory alloys such as, but not limited to, titanium or stainless steel.

Regarding FIGS. 13-16, in at least one embodiment, the bottom 105 or top surface (not shown) can be further functionalized with structural supports similar to structural supports 1703 of FIG. 17 and a flat piece of metal, metal alloy, or shape memory alloy, serving a similar function as the disc 1702 of FIG. 17. In at least one embodiment, the bottom 105 of the convex surface 103 can be functionalized with one or more structural supports and the bottom 105 of the concave surface 104 can be functionalized with one or more structural supports. The one or more structural supports of the convex surface 103 and the one or more structural supports of the concave surface 104 can be the same dimensions. Alternatively, the one or more structural supports of the convex surface 103 can be larger than the one or more structural supports of the concave surface 104. The one or more structural supports of the convex surface 103 and the one or more structural supports of the concave surface 104 can be the same shape memory alloy composition or compositions. The one or more structural supports of the convex surface 103 and the one or more structural supports of the concave surface 104 can be the same shape memory alloy composition. Alternatively, the one or more structural supports of the convex surface 103 and the one or more structural supports of the concave surface 104 can be different shape memory alloy compositions such as, for example, a first alloy comprising Ti:Nb with an at % ratio of 76:24 and a second alloy comprising Ti:Nb with an at % of 75:25. The one or more structural supports of the convex surface 103 and the one or more structural supports of the concave surface 104 can alternatively be composed of different element combinations, for example Ti:Ni and Ti:Nb.

In another exemplary aspect, methods for making a shape memory alloy spinal implant are also provided. In general, the method comprises melting and homogenizing substantially pure titanium (Ti) and niobium (Nb) to form the alloy, machining the alloy into the shape of a spinal implant, and training the spinal implant.

In at least one exemplary method of making a shape memory alloy spinal a predetermined amount of substantially pure Ti and Nb are melted and homogenized to form the shape memory alloy in bulk. In at least one embodiment, the substantially pure Ti and Nb are melted by a vacuum arc melting method. After melting, the material is mixed for a predetermined period of time until the mixture is substantially homogeneous. In at least one embodiment, homogenization can be performed over a period of one minute to 72 hours. In other embodiments, homogenization can be performed over a period of one minute to one hour.

In at least one embodiment, the relative amount of substantially pure Nb and Ti used to make the shape memory alloy is predetermined such that the final composition of the alloy is characterized as including between 20-30% Nb and between 70-80% Ti respectively. In at least one embodiment, the relative amount of substantially pure Nb and Ti used to make the shape memory alloy is predetermined such that the final composition of the alloy is characterized as including between 23-27% Nb and between 73-77% Ti respectively. In at least one embodiment, the relative amount of substantially pure Nb and Ti used to make the shape memory alloy is predetermined such that the final composition of the alloy is characterized as including between 24-26% Nb and between 74-76% Ti respectively.

In at least one embodiment, the method can further include a mechanical processing procedure including hot extrusion, warm extrusion, cold extrusion, rolling, swaging, equal channel angular pressing, equal channel angular extrusion and wire drawing, or combinations thereof, prior to machining. The mechanical processing procedure can be used to increase the strength and stability of the alloy. In at least one embodiment, the thermo-mechanical processing is performed at a temperature between 300-800° C. for 5 minutes to one hour. In other embodiments, the thermo-mechanical processing is performed at a temperature between 500-600° C. for 5 minutes to 30 minutes. In at least one embodiment, the thermo-mechanical processing comprises more than 10% of Von mises equivalent strain.

After formation of the bulk shape memory alloy, the alloy can be formed into the shape of a spinal implant by a machining process. In at least one embodiment, the desired shape and size of an implant can be formed by cutting and grinding (that is, abrasive cutting) a block of the alloy. The implant can also be made by milling a block of the alloy into the desired size and shape. Milling can be performed by computer numerical control (CNC) techniques. The implant can also be made by electric discharge machining (EDM). It will be understood by those skilled in the art that the above described shape memory alloys can be fabricated with any suitable machining process and the machining processes described herein are not intended to limit the scope of this disclosure.

Alternatively, after formation of the bulk shape memory alloy, the alloy can be formed into the shape of a spinal implant by a shape setting process. In the shape setting process, a plate or sheet of the shape memory alloy can be mechanically deformed into a predetermined shape at a temperature above the finish temperature of the martensite to austenite transformation. The deformed piece can then be immobilized in the desired shape (with a mold or clamps, for example), and heated to a predetermined temperature. The temperature can be above 400° C. and for a period of 30 seconds or longer. In alternate embodiments, the temperature can range from 400-600° C. for 3-5 minutes. The formed implant can then be cooled to room temperature, and the clamps/holder removed, to form the final implant. It will be understood by those skilled in the art that the above described shape memory alloys can be fabricated with any suitable shape setting process and the shape setting processes described herein are not intended to limit the scope of this disclosure.

In at least one embodiment, the formed spinal implant can be subjected to a heat treating process after machining. The heat treatment process can be performed over 150-800° C. from 5 minutes to 60 minutes followed by a water quench. The heat treatment process can be used to fine-tune the transformation temperatures of the implant as well as improve strength and stability. After the machining process or the optional heat treating process, the spinal implant can be trained to have predefined shape memory characteristics over a desired temperature range. Training can be performed by cyclic loading of the alloy to 1-3% strain at a temperature between 20° C. (68° F.)-50° C. (122° F.). The training can also comprise cyclic loading the spinal implant to strains larger than 0.5% strain in the austenite phase at a temperature below about 150° C. The cyclic loading can be accomplished over a plurality of loading and unloading cycles. In at least one embodiment, the number loading cycles can be between 1 and 1000. In other embodiments, the number of loading cycles can be between 100 and 1000.

In another exemplary aspect, methods for making a shape memory alloy spinal implant are also provided. In general, the method comprises melting and homogenizing substantially pure titanium (Ti), niobium (Nb) and zirconium (Zr) to form the alloy, machining the alloy into the shape of a spinal implant, and training the spinal implant.

In at least one exemplary method of making a shape memory alloy spinal implant a predetermined amount of substantially pure Ti, Nb and Zr are melted and homogenized to form the shape memory alloy in bulk. In at least one embodiment, the substantially pure Ti, Nb and Zr are melted by a vacuum arc melting method. After melting, the material is mixed for a predetermined period of time until the mixture is substantially homogeneous. In at least one embodiment, homogenization can be performed over a period of one minute to 72 hours. In other embodiments, homogenization can be performed over a period of one minute to one hour.

In at least one embodiment, the relative amount of substantially pure Nb, Ti and Zr used to make the shape memory alloy is predetermined such that the final composition of the alloy is characterized as including between 18-26% Nb, 70-80% Ti and 4-12% Zr respectively. In other embodiments, the relative amount of substantially pure Nb, Ti and Zr used to make the shape memory alloy is predetermined such that the final composition of the alloy is characterized as including between 20-24% Nb, between 70-80% Ti and between 6-10% Zr respectively.

In at least one example, the method can further include a mechanical processing procedure including hot extrusion, warm extrusion, cold extrusion, rolling, swaging, equal channel angular pressing, equal channel angular extrusion and wire drawing, or combinations thereof, prior to machining. The mechanical processing procedure can be used to increase the strength and stability of the alloy. In at least one embodiment, the thermo-mechanical processing is performed at a temperature between 300-800° C. for 5 minutes to one hour. In other embodiments, the thermo-mechanical processing is performed at a temperature between 500-600° C. for 5 minutes to 30 minutes. In at least one embodiment, the thermo-mechanical processing comprises more than 10% of Von mises equivalent strain.

After formation of the bulk shape memory alloy, the alloy can be formed into the shape of a spinal implant by a machining process. In at least one embodiment, the desired shape and size of an implant can be formed by cutting and grinding (that is, abrasive cutting) a block of the alloy. The implant can also be made by milling a block of the alloy into the desired size and shape. Milling can be performed by computer numerical control (CNC) techniques. The implant can also be made by electric discharge machining (EDM). It will be understood by those skilled in the art that the above described shape memory alloys can be fabricated with any suitable machining process and the machining processes described herein are not intended to limit the scope of this disclosure.

Alternatively, after formation of the bulk shape memory alloy, the alloy can be formed into the shape of a spinal implant by a shape setting process. In the shape setting process, a plate or sheet of the shape memory alloy can be mechanically deformed into a predetermined shape at a temperature above the finish temperature of the martensite to austenite transformation. The deformed piece can then be immobilized in the desired shape (with a mold or clamps, for example), and heated to a predetermined temperature. The temperature can be above 400° C. and for a period of 30 seconds or longer. In alternate embodiments, the temperature can range from 400-600° C. for 3-5 minutes. The formed implant can then be cooled to room temperature, and the clamps/holder removed, to form the final implant. It will be understood by those skilled in the art that the above described shape memory alloys can be fabricated with any suitable shape setting process and the shape setting processes described herein are not intended to limit the scope of this disclosure.

In at least one embodiment, the formed spinal implant is subjected to a heat treating process after machining. The heat treatment process can be performed over 150-800° C. from 5 minutes to 60 minutes followed by a water quench. The heat treatment process can be used to fine-tune the transformation temperatures of the implant as well as improve strength and stability.

After the machining process or the optional heat treating process, the spinal implant can be trained to have predefined shape memory characteristics over a desired temperature range. Training can be performed by cyclic loading of the alloy to 1-3% strain at a temperature between 20° C. (68° F.)-50° C. (122° F.). The training can also comprise cyclic loading the spinal implant to strains larger than 0.5% strain in the austenite phase at a temperature below about 150° C. The cyclic loading can be accomplished over a plurality of loading and unloading cycles. In at least one embodiment, the number loading cycles can be between 1 and 1000. In other embodiments, the number of loading cycles can be between 100 and 1000.

In another exemplary aspect, methods for making a shape memory alloy spinal implant are also provided. In general, the method comprises melting and homogenizing substantially pure titanium (Ti) and niobium (Nb) with titanium dioxide (Ti0₂) to form the alloy, machining the alloy into the shape of a spinal implant, and training the spinal implant. In at least one example, the method can further include a mechanical processing procedure including hot extrusion, warm extrusion, cold extrusion, rolling, swaging, equal channel angular pressing, equal channel angular extrusion and wire drawing, or combinations thereof, prior to machining. The mechanical processing procedure can be used to increase the strength and stability of the alloy.

In at least one exemplary method of making a shape memory alloy spinal implant a predetermined amount of substantially pure Ti and Nb and Ti0₂ are melted and homogenized to form the shape memory alloy in bulk. In at least one embodiment, the substantially pure Ti and Nb and Ti0₂ are melted by a vacuum arc melting method. After melting, the melted material is mixed for a predetermined period of time until the mixture is substantially homogeneous. In at least one embodiment, homogenization can be performed over a period of one minute to 72 hours. In other embodiments, homogenization can be performed over a period of one minute to one hour.

In at least one embodiment, the relative amount of substantially pure Ti and Nb and Ti0₂ used to make the shape memory alloy is predetermined such that the final composition of the alloy is characterized as including between 20-30% Nb, 70-80% Ti and 0.1-2% O respectively. In other embodiments, the relative amount of substantially pure Ti and Nb and Ti0₂ used to make the shape memory alloy is predetermined such that the final composition of the alloy is characterized as including between 20-30% Nb, between 70-80% Ti and between 0.5-1.5%

In other embodiments, the relative amount of substantially pure Ti and Nb and Ti0₂ used to make the shape memory alloy is predetermined such that the final composition of the alloy is characterized as including between 20-30% Nb, between 70-80% Ti and about 1%.

In at least one example, the method can further include a thermo-mechanical processing procedure including hot extrusion, warm extrusion, cold extrusion, rolling, swaging, equal channel angular pressing, equal channel angular extrusion and wire drawing, or combinations thereof, prior to machining. The mechanical processing procedure can be used to increase the strength and stability of the alloy. In at least one embodiment the thermo-mechanical processing is performed at a temperature between 300-800° C. for 5 minutes to one hour. In other embodiment the thermo-mechanical processing is performed at a temperature between 500-600° C. for 5 minutes to 30 minutes. In at least one embodiment, the thermo-mechanical processing comprises more than 10% of Von mises equivalent strain.

After formation of the bulk shape memory alloy, the alloy can be formed into the shape of a spinal implant by a machining process. In at least one embodiment, the desired shape and size of an implant can be formed by cutting and grinding (that is, abrasive cutting) a block of the alloy. The implant can also be made by milling a block of the alloy into the desired size and shape. Alternatively, milling can be performed by computer numerical control (CNC) techniques. The implant can also be made by electric discharge machining (EDM). It will be understood by those skilled in the art that the above described shape memory alloys can be fabricated with any suitable machining process and the machining processes described herein are not intended to limit the scope of this disclosure.

After formation of the bulk shape memory alloy, the alloy can be formed into the shape of a spinal implant by a shape setting process. In the shape setting process, a plate or sheet of the shape memory alloy can be mechanically deformed into a predetermined shape at a temperature above the finish temperature of the martensite to austenite transformation. The deformed piece can then be immobilized in the desired shape (with a mold or clamps, for example), and heated to a predetermined temperature. The temperature can be above 400° C. and for a period of 30 seconds or longer. In alternate embodiments, the temperature can range from 400-600° C. for 3-5 minutes. The formed implant can then be cooled to room temperature, and the clamps/holder removed, to form the final implant. It will be understood by those skilled in the art that the above described shape memory alloys can be fabricated with any suitable shape setting process and the shape setting processes described herein are not intended to limit the scope of this disclosure.

In at least one embodiment, the formed spinal implant can then subjected to a heat treating process. The heat treatment process can be performed over 150° C. to 800° C. from 5 minutes to 60 minutes followed by a water quench. The heat treatment process can be used to fine-tune the transformation temperatures of the implant as well as improve strength and stability. After the machining process or the optional heat treating process, the spinal implant can be trained to have predefined shape memory characteristics, over a desired temperature range. Training can be performed by cyclic loading of the alloy to 1-3% strain at a temperature between 20° C. (68° F.)-50° C. (122° F.). The training can also comprise cyclic loading the spinal implant to strains larger than 0.5% strain in the austenite phase at a temperature below about 150° C. The cyclic loading can be accomplished over a plurality of loading and unloading cycles. In at least one embodiment, the number loading cycles can be between 1 and 1000. In other embodiments, the number of loading cycles can be between 100 and 1000.

In another exemplary aspect, methods for making a shape memory alloy spinal implant are also provided. In general, the method comprises melting and homogenizing substantially pure titanium (Ti) and niobium (Nb) with titanium nitride (TiN) to form the alloy, machining the alloy into the shape of a spinal implant, and training the spinal implant. In at least one example, the method can further include a thermo-mechanical processing procedure including hot extrusion, warm extrusion, cold extrusion, rolling, swaging, equal channel angular pressing, equal channel angular extrusion and wire drawing, or combinations thereof, prior to machining. The thermo-mechanical processing procedure can be used to increase the strength and stability of the alloy.

In at least one exemplary method of making a shape memory alloy spinal implant a predetermined amount of substantially pure Ti and Nb and TiN are melted and homogenized to form the shape memory alloy in bulk. In at least one embodiment, the substantially pure Ti and Nb and TiN are melted by a vacuum arc melting method. After melting, the material is mixed for a predetermined period of time until the mixture is substantially homogeneous. In at least one embodiment, homogenization can be performed over a period of one minute to 72 hours. In other embodiments, homogenization can be performed over a period of one minute to one hour.

In at least one embodiment, the relative amount of substantially pure Ti and Nb and TiN used to make the shape memory alloy is predetermined such that the final composition of the alloy is characterized as including between 20-30% Nb, 70-80% Ti and 0.1-2% N respectively. In other embodiments, the relative amount of substantially pure Ti and Nb and Ti0₂ used to make the shape memory alloy is predetermined such that the final composition of the alloy is characterized as including between 20-30% Nb, between 70-80% Ti and between 0.5-1.5% N. In other embodiments, the relative amount of substantially pure Ti and Nb and Ti0₂ used to make the shape memory alloy is predetermined such that the final composition of the alloy is characterized as including between 20-30% Nb, between 70-80% Ti and about 1% N.

In at least one example, the method can further include a mechanical processing procedure including hot extrusion, warm extrusion, cold extrusion, rolling, swaging, equal channel angular pressing, equal channel angular extrusion and wire drawing, or combinations thereof, prior to machining. The mechanical processing procedure can be used to increase the strength and stability of the alloy. In at least one embodiment, the thermo-mechanical processing is performed at a temperature between 300-800° C. for 5 minutes to one hour. In other embodiments, the thermo-mechanical processing is performed at a temperature between 500-600° C. for 5 minutes to 30 minutes. In at least one embodiment, the thermo-mechanical processing comprises more than 10% of Von mises equivalent strain.

After formation of the bulk shape memory alloy, the alloy can be formed into the shape of a spinal implant by a machining process. In at least one embodiment, the desired shape and size of an implant can be formed by cutting and grinding (that is, abrasive cutting) a block of the alloy. The implant can also be made by milling a block of the alloy into the desired size and shape. Alternatively, milling can be performed by computer numerical control (CNC) techniques. The implant can also be made by electric discharge machining (EDM). It will be understood by those skilled in the art that the above described shape memory alloys can be fabricated with any suitable machining process and the machining processes described herein are not intended to limit the scope of this disclosure.

Alternatively, after formation of the bulk shape memory alloy, the alloy can be formed into the shape of a spinal implant by a shape setting process. In the shape setting process, a plate or sheet of the shape memory alloy can be mechanically deformed into a predetermined shape at a temperature above the finish temperature of the martensite to austenite transformation. The deformed piece can then be immobilized in the desired shape (with a mold or clamps, for example), and heated to a predetermined temperature. The temperature can be above 400° C. and for a period of 30 seconds or longer. In alternate embodiments, the temperature can range from 400-600° C. for 3-5 minutes. The formed implant can then be cooled to room temperature, and the clamps/holder removed, to form the final implant. It will be understood by those skilled in the art that the above described shape memory alloys can be fabricated with any suitable shape setting process and the shape setting processes described herein are not intended to limit the scope of this disclosure.

In at least one embodiment, the formed spinal implant is then subjected to a heat treating process. The heat treatment process can be performed over 150° C. to 800° C. from 5 minutes to 60 minutes followed by a water quench. The heat treatment process can be used to fine-tune the transformation temperatures of the implant as well as improve strength and stability.

After the machining process or the optional heat treating process, the spinal implant can be trained to have predefined shape memory characteristics over a desired temperature range. Training can be performed by cyclic loading of the alloy to 1-3% strain at a temperature between 20° C. (68° F.)-50° C. (122° F.). The training can also comprise cyclic loading the spinal implant to strains larger than 0.5% strain in the austenite phase at a temperature below about 150° C. The cyclic loading can be accomplished over a plurality of loading and unloading cycles. In at least one embodiment, the number loading cycles can be between 1 and 1000. In other embodiments, the number of loading cycles can be between 100 and 1000.

In another exemplary aspect, methods for making a shape memory alloy spinal implant are also provided. In general, the method comprises melting and homogenizing substantially pure titanium (Ti) and nickel (Ni) to form the alloy, machining the alloy into the shape of a spinal implant, and training the spinal implant. In at least one example, the method can further include a thermo-mechanical processing procedure including hot extrusion, warm extrusion, cold extrusion, rolling, swaging, equal channel angular pressing, equal channel angular extrusion and wire drawing, or combinations thereof, prior to machining. The thermo-mechanical processing procedure can be used to increase the strength and stability of the alloy.

In at least one exemplary method of making a shape memory alloy spinal implant a predetermined amount of substantially pure Ti and Ni are melted and homogenized to form the shape memory alloy in bulk. In at least one embodiment, the substantially pure Ti and Ni are melted by a vacuum arc melting method. After melting, the melted material is mixed for a predetermined period of time until the mixture is substantially homogeneous. In at least one embodiment, homogenization can be performed over a period of one minute to 72 hours. In other embodiments, homogenization can be performed over a period of one minute to one hour.

In at least one embodiment, the relative amount of substantially pure Ni and Ti used to make the shape memory alloy is predetermined such that the final composition of the alloy is characterized as including between 48-55% Ni and between 45-52% Ti respectively. In at least one embodiment, the relative amount of substantially pure Ni and Ti used to make the shape memory alloy is predetermined such that the final composition of the alloy is characterized as including between 48-51% Ni and between 49-52% Ti respectively. In at least one embodiment, the relative amount of substantially pure Nb and Ti used to make the shape memory alloy is predetermined such that the final composition of the alloy is characterized as including between 50.1-50.9% Ni and between 49.1-49.9% Ti respectively.

In at least one example, the method can further include a mechanical processing procedure including hot extrusion, warm extrusion, cold extrusion, rolling, swaging, equal channel angular pressing, equal channel angular extrusion and wire drawing, or combinations thereof, prior to machining. The mechanical processing procedure can be used to increase the strength and stability of the alloy. In at least one embodiment, the thermo-mechanical processing is performed at a temperature between 300-800° C. for 5 minutes to one hour. In other embodiments, the thermo-mechanical processing is performed at a temperature between 500-600° C. for 5 minutes to 30 minutes. In at least one embodiment, the thermo-mechanical processing comprises more than 10% of Von mises equivalent strain.

After formation of the bulk shape memory alloy, the alloy can be formed into the shape of a spinal implant by a machining process. In at least one embodiment, the desired shape and size of an implant can be formed by cutting and grinding (that is, abrasive cutting) a block of the alloy. The implant can also be made by milling a block of the alloy into the desired size and shape. Milling can be performed by computer numerical control (CNC) techniques. The implant can also be made by electric discharge machining (EDM). It will be understood by those skilled in the art that the above described shape memory alloys can be fabricated with any suitable machining process and the machining processes described herein are not intended to limit the scope of this disclosure.

Alternatively, after formation of the bulk shape memory alloy, the alloy can be formed into the shape of a spinal implant by a shape setting process. In the shape setting process, a plate or sheet of the shape memory alloy can be mechanically deformed into a predetermined shape at a temperature above the finish temperature of the martensite to austenite transformation. The deformed piece can then be immobilized in the desired shape (with a mold or clamps, for example), and heated to a predetermined temperature. The temperature can be above 400° C. and for a period of 30 seconds or longer. In alternate embodiments, the temperature can range from 400-600° C. for 3-5 minutes. The formed implant can then be cooled to room temperature, and the clamps/holder removed, to form the final implant. It will be understood by those skilled in the art that the above described shape memory alloys can be fabricated with any suitable shape setting process and the shape setting processes described herein are not intended to limit the scope of this disclosure.

In at least one embodiment, the formed spinal implant is then subjected to a heat treating process after machining. The heat treatment process can be performed over 200° C. to 500° C. from 15 minutes to 100 hours followed by a water quench. The heat treatment process can be used to fine-tune the transformation temperatures of the implant as well as improve strength and stability.

After the machining process or the optional heat treating process, the spinal implant can be trained to have predefined shape memory characteristics over a desired temperature range. Training can be performed by cyclic loading of the alloy to 1-8% strain at a temperature between 20° C. (68° F.)-50° C. (122° F.). The training can also comprise cyclic loading the spinal implant to strains larger than 0.5% strain in the austenite phase at a temperature below about 150° C. The cyclic loading can be accomplished over a plurality of loading and unloading cycles. In at least one embodiment, the number loading cycles can be between 1 and 1000. In other embodiments, the number of loading cycles can be between 100 and 1000.

In another exemplary aspect, methods for making a spinal implant comprising more than one shape memory alloy are also provided. In general, the method comprises forming more than one alloy wherein each alloy has a different composition and temperature dependent shape memory characteristics, connecting the more than one shape memory alloys together, machining the connected shape metal alloys to form a spinal implant, and training the spinal implant as above with respect to the previously described embodiments. The more than one shape memory alloy can be directly connected by, for example, laser welding. The more than one shape memory alloys can also be indirectly connected by fusing each to another material such as, for example, a plate or strip of pure titanium or one or more biocompatible adhesives. In one example, disclosed herein is a spinal implant comprising more than one shape memory alloy such that different regions of the implant, corresponding to different shape memory alloys, undergo shape memory changes over different temperature ranges. The implant can be fabricated such that, for example, a first portion of the implant, corresponding to a first shape memory alloy, can expand over a first temperature range in a substantially perpendicular direction relative to the spinal column and a second portion of the implant, corresponding to a second shape memory alloy, can expand over a second temperature range higher than the first temperature range in a substantially parallel direction relative to the spinal column. Here, the first and second shape memory alloys can be composed of the same chemical elements, for example Ti:Nb, but each having a different composition such as for example, a first alloy comprising Ti:Nb with an at % of 76:24 and a second alloy comprising Ti:Nb with an at % of 75:25. The first and second shape memory alloys can alternatively be composed of different elements, for example Ti:Ni and Ti:Nb. It will be understood by those skilled in the art that suitable ranges include ranges include any combination of the specified atomic percentages even if the specific combination is not listed herewith. It will also be understood by those skilled in the art that any of the above described shape memory alloys can be used to form a spinal implant comprising more than one shape memory alloy.

In another exemplary aspect, processes for implanting a spinal implant are also provided. In general, the spinal implant may be inserted into a vertebral cavity corresponding to the removed compromised spinal. The spinal implant is kept at a low temperature relative to room temperature, exhibits a shrunken first size and shape and is introduced into a vertebral cavity of a patient. Upon introduction of the spinal implant, the implant will expand to a larger, predetermined, second size and shape in response to the body temperature of the patient.

Prior to, or during, expansion of the spinal implant, a suitable osteogenic material and/or therapeutic composition can be incorporated with the spinal implant. Suitable osteogenic materials can include, for example, autograft, allograft, xenograft, demineralized bone, synthetic and natural bonegraft substitutes, such as bioceramics, polymers and osteoinducive factors. These materials can be provided into or onto roughened or corrugated surfaces of the spinal implant, or recesses or apertures running therethrough. A separate carrier such as, for example, collagen-based carriers, bioceramic materials, calcium phosphate, hydroxyapatite, or any combination thereof, can be used to hold the osteogenic materials in their desired location. The carriers may also be partially comprised of therapeutic or infection resistant agents. The carriers may also be partially comprised of an effective amount bone morphogenic protein, transforming growth factor 1, insulin-like growth factor 1, platelet-derived growth factor, fibroblast growth factor, LIM mineralization protein (LMP), and any combinations thereof.

The method of implantation into a patient can be, but is not limited to, Anterior; Lumbar Interbody Fusion (ALIF) Surgery, Posterior Lumbar Interbody Fusion (PLIF) Surgery; Transforaminal Lumbar Interbody Fusion (TLIF) Surgery, eXtreme Lateral Interbody Fusion (XLIF) Surgery, or any other suitable implantation technique.

Exemplary embodiments are disclosed and variations, combinations, and/or modifications of the embodiment(s) and/or features of the embodiment(s) made by a person having ordinary skill in the art are within the scope of the disclosure. Alternative embodiments that result from combining, integrating, and/or omitting features of the embodiment(s) are also within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, Rₗ, and an upper limit, Rᵤ, is disclosed, any number falling within the range is specifically disclosed. In particular, the following numbers within the range are specifically disclosed: R=Rₗ+k*(Rᵤ−Rₗ), wherein k is a variable ranging from 1 percent to 100 percent with a 1 percent increment, i.e., k is 1 percent, 2 percent, 3 percent, 4 percent, 5 percent, . . . , 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. Use of broader terms such as “comprises,” “includes,” and “having” should be understood to provide support for narrower terms such as “consisting of,” “consisting essentially of,” and “comprised substantially of.” Terms including “substantially” may be understood to be a functional definition to describe a property or feature that may vary between patients, and which may be within 5%, 7%, or 10% of each other, Accordingly, the scope of protection is not limited by the description set out above but is defined by the claims that follow, that scope including all equivalents of the subject matter of the claims. Each and every claim is incorporated into the specification as further disclosure, and the claims are exemplary embodiment(s) of the present invention.

While preferred embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. For example, the relative dimensions of various parts, the materials from which the various parts are made, and other parameters can be varied. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

1. An implant assembly comprising:
niobium (Nb); and
titanium (Ti), wherein the implant assembly is configured to expand a first predetermined amount along substantially a first plane at a first temperature and to expand a second predetermined amount along substantially a second plane at a second temperature, wherein the first plane is substantially perpendicular to the second plane, and wherein the second temperature is higher than the first temperature.

2. The implant assembly of claim 1, wherein the expansion of the first plane and the second plane are configured to result in an expanded implant assembly configured to conformance fit to a cavity of a patient.

3. The implant assembly of claim 1, wherein the implant assembly comprises at least one shape memory alloy comprising an atomic % of Nb between about 20-30% and an atomic % of Ti between 70-80%.

4. The implant assembly of claim 1, wherein the at least one shape memory alloy further comprises zirconium (Zr).

5. The implant assembly of claim 5, wherein the at least one shape memory alloy comprises an atomic % of Nb between 18-26%, an atomic percentage of Ti between 70-80%, and an atomic % of Zr between 4-12%.

6. The implant assembly of claim 6, wherein the at least one shape memory alloy further comprises an atomic % of oxygen (0) between 0.1-2%.

7. The implant assembly of claim 6, wherein the at least one shape memory alloy further comprises an atomic % of nitrogen (N) between 0.1-2%.

8. The implant assembly of claim 1, wherein the implant further comprises a second shape memory alloy, wherein a composition of the at least one shape memory alloy is different from a composition of the second shape memory alloy.

9. The implant assembly of claim 1, wherein the implant further comprises a second shape memory alloy, wherein a shape-setting process of the at least one shape memory alloy is different from a shape-setting process of the second shape memory alloy.

10. The implant assembly of claim 1, wherein the expansion of the at least one shape memory alloy is tailored based on a composition and a processing of the at least one shape memory alloy.

11. An implant assembly comprising:
a first portion comprising a first shape memory alloy; and
a second portion comprising a second shape memory alloy;
where in each of the first shape memory alloy and the second shape memory alloy comprise nickel (Ni) and titanium (Ti); and
wherein the first shape memory alloy is configured to expand a first predetermined amount along substantially a first plane at a first temperature,
wherein the second shape memory alloy is configured to expand a second predetermined amount along substantially a second plane at a second temperature; and
wherein the first plane and the second plane are substantially perpendicular, and wherein the second temperature is higher than the first temperature.

12. The implant assembly of claim 11 wherein the shape memory alloy comprises an atomic % of Ni between 48-55% and an atomic % of Ti between 45-52%.

13. The implant assembly of claim 11, wherein the first shape memory alloy comprises at least one of a different composition or a different shape-setting than the second shape memory alloy, and wherein the first portion is one of directly and indirectly coupled to the second portion.

14. The implant assembly of claim 11, wherein the first shape memory alloy is configured to expand a third predetermined amount along substantially the second plane at the first temperature, and wherein the second shape memory alloy is configured to expand a fourth predetermined amount along substantially the first plane at the second temperature.

15. A method of making a shape memory alloy implant assembly comprising:
melting and homogenizing titanium (Ti) and niobium (Nb) to form a first shape memory alloy and a second shape memory alloy;
forming a first portion of an implant assembly from the first shape memory alloy;
forming a second portion of the implant assembly from the second shape memory alloy;
shape-setting the first and the second shape memory alloys, wherein, subsequent to the shape-setting the first shape memory alloy is configured to expand a first predetermined amount along substantially a first plane at a first temperature and the second shape memory alloy is configured to expand a second predetermined amount along substantially a second plane at a second temperature, wherein the first plane and the second plane are substantially perpendicular, and wherein the second temperature is higher than the first temperature.

16. The method of claim 15, wherein shape-setting comprises at least one of a thermo-mechanical process and a machining process.

17. The method of claim 15, wherein the first shape memory alloy comprises a different composition than the second shape memory alloy.

18. The method of claim 15, wherein at least one of oxygen (O), titanium nitride (TiN), nitrogen (N), titanium oxide (Ti02) and zirconium (Zr) is melted and homogenized with the Ti and the Nb to form at least one of the first shape memory alloy and the second shape memory alloy.

19. The method of claim 18, wherein an atomic % of oxygen (0) between 0.1-2%, and wherein an atomic % of nitrogen (N) between 0.1-2%.

20. The method of claim 15, wherein shape-setting the first shape memory alloy comprises a first shape-setting process, wherein shape-setting the second shape memory alloy comprises a second shape-setting process, and wherein the first shape-setting process is different than the second shape setting process.
